(19) United States
(12) Patent Application Publication
(10) Pub. No.: US 2023/0046704 A1
(43) Pub. Date: Feb. 16, 2023

(54) REMOTE MONITORING AND SUPPORT OF MEDICAL DEVICES

(71) Applicant: Medtronic, Inc., Minneapolis, MN (US)

(72) Inventors: Matthew R. Yoder, Crystal, MN (US); Amruta Paritosh Dixit, Maple Grove, MN (US); Gaurav Makin, Shoreview, MN (US); Joel R. Lauer, Clearwater, MN (US); Eduardo N. Warman, Maple Grove, MN (US); Shantanu Sarkar, Roseville, MN (US); Kevin T. Ousdigian, Shoreview, MN (US); Ya-Jian Cheng, Lino Lakes, MN (US)

(21) Appl. No.: 17/402,281

(22) Filed: Aug. 13, 2021

(51) Int. Cl. A61B 5/00

(52) U.S. Cl. CPC A61B 5/7282; A61B 5/7267; A61B 5/686; A61B 5/7275

(57) ABSTRACT

This disclosure is directed to systems and techniques for detecting change in patient health and if a change in patient health is detected, direct a medical device to generate for display output indicating the detection of the change in patient health. An example medical system or technique applies a model to values of configurable settings that are programmed into detection logic of a medical device; based on the application, determine whether modified values of the configurable settings, when implemented by the detection logic, would change a determination, by the medical device, regarding whether sensed physiological activity is indicative of cardiac episode for a patient; and in response to a determination that the modified values would change the determination regarding whether the sensed physiological activity is indicative of the cardiac episode for the patient, generate output data indicative of the modified values for the configurable settings for the medical device.

### FIELD

The disclosure relates generally to medical systems and, more particularly, medical systems configured to support medical devices engaged in patient monitoring and/or treatment.

### BACKGROUND

Medical systems may monitor various data (e.g., an electrocardiogram (ECG) or a cardiac electrogram (EGM)) of a patient or a group of patients to detect changes in health. In some examples, the medical system may monitor the cardiac EGM to detect one or more types of arrhythmia, such as bradycardia, tachycardia, fibrillation, or asystole (e.g., caused by sinus pause or AV block). In some examples, the medical system may include one or more of an implantable medical device or a wearable device to collect various measurements used to detect changes in patient health. In some examples, medical systems may include one or more devices configured to deliver therapy to treat conditions. The delivery of therapy may be based on the monitored data.

### SUMMARY

Medical systems and techniques as described herein facilitate medical device functionality including any medical device engaged in patient monitoring and/or therapy. An example medical system may include a network-based computing system configured to run a service that provides the medical device with computing resources as a form of (e.g., cloud-based) operational support. While the medical device is coupled to a patient and logic circuitry (e.g., logic implemented in the medical device) is actively monitoring the patient's physiological activities (e.g., to detect cardiac maladies) and, in some cases, controlling delivery of therapy, the network-based computing system may run the service to evaluate the medical device, for example, with respect to the medical device's configuration (e.g., being indicative of performance). Based on that evaluation, the network-based computing system may determine whether to update the medical device configuration in a manner that improves its patient monitoring and/or therapy functionality.

The configuration of a medical device may be defined by a number of configurable settings. Periodically, based on a user request, and/or in response to detection of an event, the service may evaluate the current configuration of the medical device, and may return a rejection of the configuration with modified settings or return a confirmation. It should be noted that the “modified settings” described herein refer to actual values (i.e., modified values) established for the configurable settings of the medical device. For any given configurable setting, that setting's value determines how the medical device functions in at least one aspect and therefore, modifying the setting's value to a different value may result in a change to the medical device's functionality in that at least one aspect.

When the medical device receives modified settings, the medical device replaces the medical device's current/default settings with the modified settings. In some of the examples described herein, the modified settings may result in a change to the medical device's configuration (e.g., by altering the detection and/or therapy logic implemented in the medical device) and possibly, change the medical device's determinations regarding patient health. In some examples, the service may reject the configuration if implementing the modified settings causes the medical device to detect a different malady or produce an alternative diagnosis. As described herein, the service may be configured to maintain and/or determine effective settings for the configuration of the medical device, for example, to achieve a certain performance level.

In addition to potentially ensuring the patient is equipped with a properly configured medical device for providing medical care, the techniques described herein may reduce clinician burden in some areas. By updating the configuration of the medical device with effective settings, the service may allow the patient's clinician to spend more of their time on the patient's care without having to determine how to properly configure the patient's device themselves. Having the service configure the medical device to operate effectively may also result in lower or non-existent malfunction rates. In view of the above, the present disclosure describes a technological improvement or a technical solution that is integrated into a practical application.

In one example, a medical system comprising: processing circuitry configured to: apply a model to values of configurable settings that are programmed into detection logic of a medical device; based on the application of the model, determine whether modified values of the configurable settings, when implemented by the detection logic, would change a determination, by the medical device, regarding whether sensed physiological activity is indicative of cardiac episode for a patient; and in response to a determination that the modified values would change the determination regarding whether the sensed physiological activity is indicative of the cardiac episode for the patient, generate output data indicative of the modified values for the configurable settings for the medical device.

In another example, a method performed by a computing device communicatively coupled to one or more medical devices, the method comprising: applying, by processing circuitry of the computing device, a model to feature data of the one or more medical devices, wherein the model is configured to calibrate one or more configurable settings of each medical device; by the processing circuitry, determining, based on the application of the model, whether to modify default or current values of the configurable settings; and in response to a determination to modify the default or current values of the configurable settings, generate output data indicative of modified values for the configurable settings for the medical device.

In another example, a non-transitory computer-readable storage medium comprising program instructions that, when executed by processing circuitry of a medical device, cause the processing circuitry to: apply a model to current or default values of configurable settings that are programmed into detection logic of a medical device, wherein the detection logic is configured to determine whether sensed physiological activity is indicative of cardiac episode for a patient; based on the application of the model, determine whether modified values of the configurable settings, when implemented by the detection logic, would change an initial detection, by the medical device, of the cardiac episode in the sensed physiological activity; and in response to a determination that the modified values would change the initial detection of the cardiac episode for the patient, generate output data indicative of a rejection of the initial detection.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF THE DRAWINGS

FIG. 1 illustrates example environment of an example medical system in conjunction with a patient, in accordance with one or more examples of the present disclosure.

FIG. 2 is a functional block diagram illustrating an example configuration of a medical device, in accordance with one or more examples of the present disclosure.

FIG. 3 is a conceptual side-view diagram illustrating an example configuration of the IMD of FIGS. 1 and 2, in accordance with one or more examples of the present disclosure.

FIG. 4 is a functional block diagram illustrating an example configuration of the external device of FIG. 1, in accordance with one or more examples of the present disclosure.

FIG. 5 is a block diagram illustrating an example system that includes an access point, a network, external computing devices, such as a server, and one or more other computing devices, which may be coupled to the medical device and external device of FIGS. 1-4, in accordance with one or more examples of the present disclosure.

FIG. 6 is a block diagram illustrating an example computing service to provide resources to medical devices of an example medical system, such as the system of FIG. 1, in accordance with one or more examples of the present disclosure.

FIG. 7 is a flow diagram illustrating an example operation for remotely monitoring medical devices in an example medical system, in accordance with one or more examples of the present disclosure.

FIG. 8 is a flow diagram illustrating an example operation by a computing service for automatically configuring medical device settings, in accordance with one or more examples of the present disclosure.

Like reference characters denote like elements throughout the description and figures.

### DETAILED DESCRIPTION

In general, medical systems according to this disclosure implement techniques for network-based support of medical device functionality. Medical devices of an example medical system (e.g., pacemakers or other therapy devices, or implantable or wearable cardiac monitoring devices) are awash in configurable settings that may be activated/deactivated or (e.g., in the case of settings having multiple possible numerical values or other values) otherwise changed (e.g., by a clinician) in different combinations. For example, the clinician may change certain settings to tailor a particular medical device's functionality to a patient (e.g., a patient's physiology) or a patient population (e.g., heart failure patients) or a patient population sub-group (e.g., heart failure patients with a certain comorbidity or any comorbidity).

Clinicians often forego manual selections for most (if not all) of these settings, and instead may rely on manufacturer default settings for the particular medical device, which may be less effective than other possible settings. Even when a clinician manually selects settings, it is possible that the clinician will program the particular medical device settings that may be less effective than other possible settings. Given the breadth of options offered by a device, there are a considerable number of considerable settings for any device and often, this presents an immense burden to the point that a non-trivial percentage of medical devices perform at a less desirable level. As described herein, the computing service (e.g., a network-based service) may run on a computing system that provides the network based support to the patient's medical device, for example, by (automatically) configuring that devices with appropriate settings, resulting in improved overall performance (e.g., in terms of accuracy and/or efficacy) for the device and overcoming/mitigating clinician burdens.

When performing a diagnosis for or delivering therapy to the patient, clinicians are often overwhelmed with tasks and avoid further complicating the diagnosis/therapy with managing the configuration of each medical device they handle for their patients. Some clinicians rely on default settings, and while a medical device may be pre-configured with suitable device settings, these device settings may be sub-optimal and may be upgraded/replaced with modified settings that are calibrated to patient's physiology and more likely to result in accurate determinations regarding patient's diagnosis/therapy. Instead of modifying the device settings to improve upon the medical device's accuracy, the clinician may choose to ignore this burden while providing medical care and simply, take into account a higher likelihood of false determinations (e.g., false positive or false negative). As a result, in a substantial number of patient cases, a medical device may be properly configured (e.g., for standard performance or even peak performance) for a vast majority of a population but may be less than optimal for a particular patient (e.g., an outlier).

To address these issues and any collateral effects to the example medical system, the techniques described herein may host the network-based service on a remote computing system that may provide device settings that most likely to result in peak performance for that medical device's type and/or for its patient's physiology, for example, in terms of specificity and sensitivity metrics. To maintain a patient's medical device that is properly configured for a patient population general operation and/or is programmed with settings modulated that patient, the network-based service may update the medical device's configuration with appropriate settings (when needed). In this manner, the techniques of this disclosure may, for example, advantageously enable improved accuracy in the detection of changes in patient health and, consequently, better evaluation of the condition of the patient. In addition or in the alternative, the techniques of this disclosure may, for example, advantageously enable improved efficacy of a therapy in treating a condition of a patient.

The techniques describe herein may configure the network-based service to provide other benefits to medical devices in the example medical system. In some examples, each medical device of the example medical system may include logic (e.g., detection logic) to perform their corresponding functionality; in some examples, various settings may be configured for each device of which some may affect their performance. In the example medical system, the remote computing system running the network-based service may receive a request for an evaluation of an example medical device's logic, for example, regarding output data (e.g., determinations) generated by the device's logic. The network-based service may be configured to (e.g., with a micro-service configured to) evaluate the medical device's logic with respect to accuracy in determining whether some patient data has a malady. As another example, the network-based service may fine-tune the accuracy evaluation to the particular patient's physiology of the medical device. This may involve a detection analysis of patient data storing the patient's physiological measurements.

Example medical devices that collect patient data and may benefit from the techniques described herein may include an implantable or wearable monitoring device, a pacemaker/defibrillator, or a ventricular assist device (VAD). The example medical device may communicate to the computing system running the network-based service the patient data in a service request, for example, for data and/or application services.

Given a service request for a pacemaker, as an example medical device, the network-based service may identify a configurable setting specifying which heart chamber (e.g., ventricle or atrial chambers) receives electrical therapy. The network-based service may determine the appropriate heart chamber, such as a left ventricle, into which an identified combination of electrodes administer the electrical therapy, for example, to correct cardiac asynchrony. Additional configurable settings (e.g., which combination of electrodes are used to deliver the electrical therapy to the particular heart chamber) may tailor the electrical therapy being administered in that heart chamber (e.g., left ventricle), for example, to a patient's physiology. Given a number of possible classes (e.g., values) for the configurable settings, the network-based service may group unique combinations of the possible classes into a set; then, to select (e.g., predict) the unique combination most likely to result in a highest performance level amongst the other unique combinations of the configurable settings, the network-based service may apply a model (e.g., a mathematical model such as a metric or a machine learning model such as a neural network) to that set and various feature data.

In a clinical (e.g., office) setting, clinicians often have trouble diagnosing certain issues in their patients and for lack of any alternative, send those patients home with a medical device (e.g., a cardiac monitoring device such as a chest strap device or a loop recorder device) that provides one or both of long-term and short-term recording. The clinician setting by its nature may inhibit an accurate diagnosis (e.g., detection) of potential issues that the patients may have such that those issues are most often diagnosed outside of the clinician setting. To that end, the above example medical device records data for various patient activities.

While some example medical devices include logic (e.g., detection logic) for determining whether recorded patient activity (e.g., cardiac activity data including cardiac events and cardiac rhythms) is indicative of a malady (e.g., a type of cardiac episode), other example medical devices are limited to recording events for offline clinical evaluation. Even if the example medical device is capable of patient monitoring and detection of one or more maladies, the example medical device may be either improperly configured or under-configured and would benefit from a post-processing analysis.

In response to a service request for an example cardiac monitoring device (or an alternative example medical device) such as any of the above-mentioned medical devices, an example network-based service may identify one or more configurable (device) settings for determining whether recorded cardiac activity data is indicative of a type of cardiac episode. Given default or current values for the one or more configurable settings, the example medical device may be unable to identify the cardiac episode (e.g., more often than not). The example network-based service may (e.g., automatically) return a service response indicating one or more modified (e.g., calibrated) values of the one or more configurable setting and in turn, a hardware/software component programs the one or more modified values into detection logic of the example cardiac monitoring device. A given patient and their clinician may benefit from more accurate and more responsive remote medical device monitoring and detection functionality, for instance, providing operational support to the clinician's effort to diagnose previously undiagnosed issues the patient may presents. Instead of or in addition to submitting the patient's cardiac activity data to the clinician, the example cardiac monitoring device may review the patient's cardiac activity data for indicia of a suspected cardiac episode. As a result, fainting episodes, racing heart rates, and a number of other example undiagnosed issues may now be diagnosed with cardiac recording devices such as the above loop recorder or other devices with minimal or no clinician involvement.

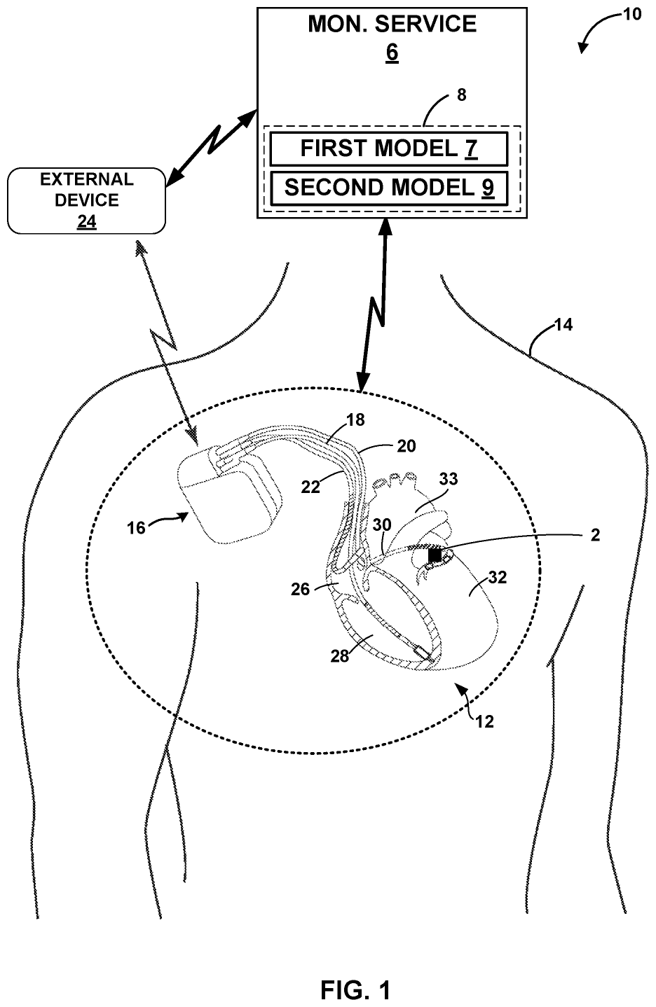

FIG. 1 illustrates example medical device system 10 in conjunction with patient 14. Medical device system 10 is an example of a medical device system that is configured to implement the example techniques described herein for estimating physiological parameter values and, in some examples, controlling the delivery of CRT to heart 12 of patient 14 based on the estimated physiological parameter values. In some examples, medical device system 10 includes an implantable medical device (IMD) 16 in communication with external device 24. In the illustrated example, IMD 16 may be coupled to leads 18, 20, and 22. IMD 16 may be, for example, an implantable pacemaker that provides electrical signals to heart 12 and senses electrical activity of heart 12 via electrodes coupled to one or more of leads 18, 20, and 22. In some examples, IMD 16 may include cardioversion and/or defibrillation capabilities.

Leads 18, 20, 22 extend into heart 12 of patient 14 to sense electrical activity of heart 12 and to deliver electrical stimulation to heart 12. In the example shown in FIG. 1, right ventricular (RV) lead 18 extends through one or more veins (not shown), the superior vena cava (not shown), and right atrium (RA) 26, and into RV 28. Left ventricular (LV) coronary sinus lead 20 extends through one or more veins, the vena cava, right atrium 26, and into the coronary sinus 30 to a region adjacent to the free wall of LV 32 of heart 12. Right atrial (RA) lead 22 extends through one or more veins and the vena cava, and into the RA 26 of heart 12.

IMD 16 may sense electrical signals attendant to the depolarization and repolarization of heart 12 via electrodes (not shown in FIG. 1) coupled to at least one of the leads 18, 20, 22. In some examples, IMD 16 may also sense electrical signals attendant to the depolarization and repolarization of heart 12 via extravascular electrodes (e.g., electrodes positioned outside the vasculature of patient 14), such as epicardial electrodes, external surface electrodes, subcutaneous electrodes, and the like. The configurations of electrodes used by IMD 16 for sensing and pacing may be unipolar or bipolar.

In some examples, IMD 16 is configured to provide CRT to heart 12. In some examples, as part of the CRT, IMD 16 is configured to deliver at least one of fusion pacing to heart 12 and biventricular pacing to heart 12. In some examples of fusion pacing, IMD 16 may deliver a pacing stimulus (e.g., a pacing pulse) to LV 32 via electrodes of lead 20, where the pacing stimulus is timed such that an evoked depolarization of LV 32 is affected in fusion with the intrinsic depolarization of RV 28, resulting in a ventricular resynchronization. In this way, the pacing pulse delivered to LV 32 (LV_{P}) may pre-excite a conduction delayed LV 32 and help fuse the activation of LV 32 with the activation of RV 28 from intrinsic conduction. The fusion of the depolarization of LV 32 and RV 28 may result in synchronous activation and contraction of LV 32 with RV 28. In the examples described herein, the fusion pacing configuration may be referred to as “left-ventricular” pacing. However, it should be understood that a fusion pacing configuration may also include right-ventricular pacing in any of the examples described.

In some examples, when IMD 16 is in a biventricular pacing configuration, IMD 16 may deliver a pacing stimulus (e.g., a pacing pulse) to RV 28 via electrodes of lead 18 and a pacing stimulus to LV 32 via electrodes of lead 20 in a manner that synchronizes activation and contraction of RV 28 and LV 32.

As discussed in further detail below, IMD 16 may be configured to adjust one or more pacing parameters based on a cardiac status of heart 12. In some examples, IMD 16 may be configured to adjust a pacing parameter by delivering electrical stimulation therapy to heart 12 according to either a fusion pacing configuration or a biventricular pacing configuration.

In some examples, the CRT provided by IMD 16 may be useful for maintaining the cardiac rhythm in patient 14 with a conduction dysfunction, which may result when the natural electrical activation system of heart 12 is disrupted. The natural electrical activation system of a human heart 12 involves several sequential conduction pathways starting with the sino-atrial (SA) node, and continuing through the atrial conduction pathways of Bachmann's bundle and internodal tracts at the atrial level, followed by the atrio-ventricular (AV) node, Common Bundle of His, right and left bundle branches, and a final distribution to the distal myocardial terminals via the Purkinje fiber network.

In a normal electrical activation sequence, the cardiac cycle commences with the generation of a depolarization wave at the SA Node in the wall of RA 26. The depolarization wave is transmitted through the atrial conduction pathways of Bachmann's Bundle and the Internodal Tracts at the atrial level into the LA 33 septum. When the atrial depolarization wave has reached the AV node, the atrial septum, and the furthest walls of the right and left atria 26, 33, respectively, the atria 26, 33 may contract as a result of the electrical activation. The aggregate right atrial and left atrial depolarization wave appears as the P-wave of the PQRST complex of an electrical cardiac signal, such as a cardiac electrogram (EGM) or electrocardiogram (ECG). When the amplitude of the atrial depolarization wave passing between a pair of unipolar or bipolar pace/sense electrodes located on or adjacent RA 26 and/or LA 33 exceeds a threshold, it is detected as a sensed P-wave. The sensed P-wave may also be referred to as an atrial sensing event, or an RA sensing event (RAs). Similarly, a P-wave sensed in the LA 33 may be referred to as an atrial sensing event or an LA sensing event (LAs).

During or after the atrial contractions, the AV node distributes the depolarization wave inferiorly down the Bundle of His in the intraventricular septum. The depolarization wave may travel to the apical region of heart 12 and then superiorly though the Purkinje Fiber network. The aggregate right ventricular and left ventricular depolarization wave and the subsequent T-wave accompanying re-polarization of the depolarized myocardium may appear as the QRST portion of the PQRST cardiac cycle complex. When the amplitude of the QRS ventricular depolarization wave passing between a bipolar or unipolar pace/sense electrode pair located on or adjacent RV 28 and/or LV 32 exceeds a threshold, it is detected as a sensed R-wave. The sensed R-wave may also be referred to as a ventricular sensing event, an RV sensing event (RVs), or an LV sensing event (LVs) depending upon the ventricle in which the electrodes of one or more of leads 18, 20, 22 are configured to sense in a particular case.

Some patients, such as patients with congestive heart failure or cardiomyopathies, may have left ventricular dysfunction, whereby the normal electrical activation sequence through heart 12 is compromised within LV 32. In a patient with left ventricular dysfunction, the normal electrical activation sequence through the heart of the patient becomes disrupted. For example, patients may experience an intra-atrial conduction defect, such as intra-atrial block. Intra-atrial block is a condition in which the atrial activation is delayed because of conduction delays between RA 26 to LA 33.

As another example, a patient with left ventricular dysfunction may experience an interventricular conduction defect, such as left bundle branch block (LBBB) and/or right bundle branch block (RBBB). In LBBB and RBBB, the activation signals are not conducted in a normal fashion along the right or left bundle branches respectively. Thus, in patients with bundle branch block, the activation of either RV 28 or LV 32 is delayed with respect to the other ventricle, causing asynchrony between the depolarization of the right and left ventricles. This asynchrony may result is decreased mechanical performance of the heart, which may be reflected in measures such as ejection fraction, stroke volume, LV pressure, and derivatives of LV pressure.

CRT delivered by IMD 16 may help alleviate heart failure conditions by restoring synchronous depolarization and contraction of one or more chambers of heart 12. In some cases, the fusion pacing of heart 12 described herein enhances mechanical performance of the heart of a patient by improving the synchrony with which RV 28 and LV 32 depolarize and contract. In some examples, measures of mechanical performance of the heart may be used to evaluate the efficacy of CRT and, in some cases, as feedback for modification of one or more parameters of CRT, such as A-V intervals or selections of which electrodes are used to deliver the CRT. However, measures of the mechanical performance of the left side of the heart tend to be invasive and thus disfavored for chronic monitoring of CRT efficacy. The techniques of this disclosure may allow system 10, e.g., IMD 16, to estimate such measures to determine efficacy of CRT and allow feedback control of CRT parameters.

In some examples, IMD 16 also provides defibrillation therapy and/or cardioversion therapy via electrodes located on at least one of the leads 18, 20, 22. IMD 16 may detect arrhythmia of heart 12, such as fibrillation of ventricles 28 and 32, and deliver defibrillation therapy to heart 12 in the form of electrical shocks. In some examples, IMD 16 is programmed to deliver a progression of therapies, e.g., shocks with increasing energy levels, until a fibrillation of heart 12 is stopped. In examples in which IMD 16 provides defibrillation therapy and/or cardioversion therapy, IMD 16 may detect fibrillation by employing any one or more fibrillation detection techniques known in the art.

External device 24 may be a computing device with a display viewable by a user and include an interface that receives input from a user. In some examples, external device 24 may be a notebook computer, tablet computer, workstation, one or more servers, cellular phone, personal digital assistant, or another computing device that may run an application that enables the computing device to interact with IMD 16. The user interface may include, for example, a keypad and a display, which may for example, be a cathode ray tube (CRT) display, a liquid crystal display (LCD) or light emitting diode (LED) display. The keypad may take the form of an alphanumeric keypad or a reduced set of keys associated with particular functions. External device 24 can additionally or alternatively include a peripheral pointing device, such as a mouse, via which a user may interact with the user interface. In some embodiments, a display of external device 24 may include a touch screen display, and a user may interact with external device 24 via the display.

A user, such as a physician, technician, or other clinician, may interact with external device 24 to communicate with IMD 16. For example, the user may interact with external device 24 to retrieve physiological or diagnostic information from IMD 16. A user may also interact with external device 24 to program IMD 16, e.g., to select values for operational parameters of the IMD.

For example, the user may use external device 24 to retrieve information from IMD 16 regarding the rhythm of heart 12, trends therein over time, or arrhythmia episodes. As another example, the user may use external device 24 to retrieve information from IMD 16 regarding other sensed physiological parameters of patient 14, such as sensed electrical activity, activity, posture, respiration, thoracic impedance, or other data related to the techniques described herein from IMD 16. As another example, the user may use external device 24 to retrieve information from IMD 16 regarding the performance or integrity of IMD 16 or other components of system 10, such as leads 18, 20, and 22, or a power source of IMD 16. In such examples, physiological parameters of patient 14 and data regarding IMD 16 may be stored in a memory of IMD 16 for retrieval by the user.

The user may use external device 24 to program a therapy progression, select electrodes used to deliver defibrillation pulses, select waveforms for the defibrillation pulse, or select or configure a fibrillation detection algorithm for IMD 16. The user may also use external device 24 to program aspects of other therapies provided by IMD 16, such as cardioversion, CRT, or pacing therapies. In some examples, the user may activate certain features of IMD 16 by entering a single command via external device 24, such as depression of a single key or combination of keys of a keypad or a single point-and-select action with a pointing device.

Monitoring service 6, IMD 16, external device 24 and, optionally, another computing device (not illustrated in FIG. 1) may communicate via wireless communication using any techniques known in the art. External device 24, for example, may communicate via near-field communication technologies (e.g., inductive coupling, NFC or other communication technologies operable at ranges less than 10-20 cm) and far-field communication technologies (e.g., radiofrequency (RF) telemetry according to the 802.11 or Bluetooth® specification sets, or other communication technologies operable at ranges greater than near-field communication technologies). An example of a viable communication technique may include radiofrequency (RF) telemetry, for example, which may be an RF link established via an antenna according to Bluetooth, WiFi, or medical implant communication service (MICS), though other techniques are also contemplated. In some examples, external device 24 may include a programming head that may be placed proximate to the patient's body near the IMD 16 implant site in order to improve the quality or security of communication between IMD 16 and external device 24.

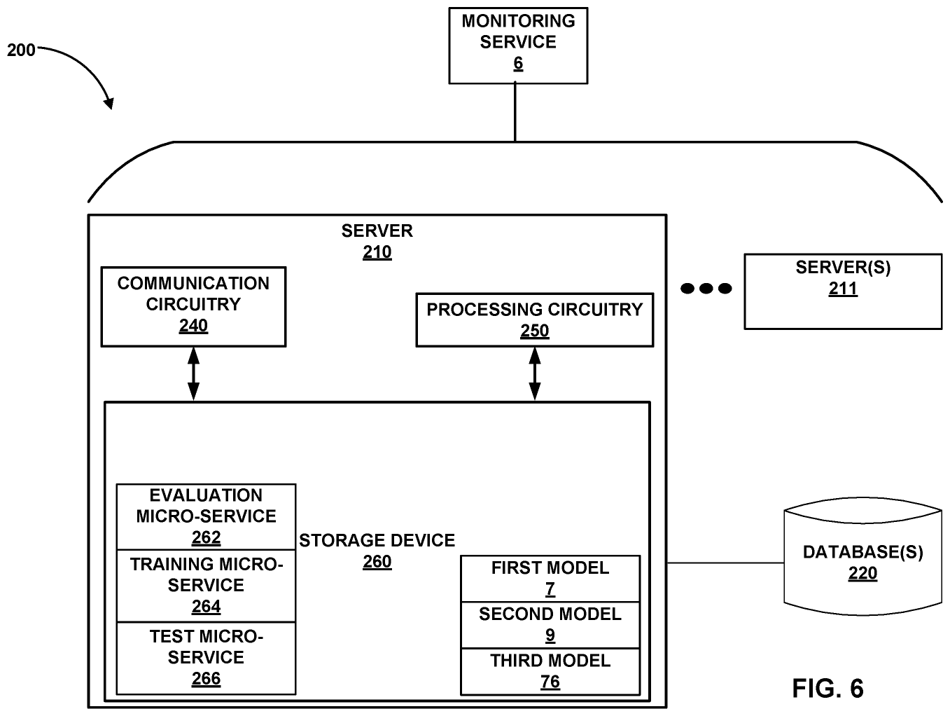

Medical system 10 includes a computing system communicatively coupled, over a network connection, to one or more medical devices including IMD 16 and, via wireless communication protocols, exchanges various data with IMD 16. Processing circuitry of medical system 10 may include one or more processors that are configured to, via communication circuitry, receive various patient data from IMD 16 and, possibly, external device 24. Designated by medical system 10 for IMD 16 and, perhaps, at least one other medical device, the computing system may operate as monitoring service 6, a network-based service, from which device calibration is accessible. While a number of computing systems may combine to form example medical system 10, at least one remote computing device may be configured run the network-based service as illustrated in FIG. 6. In general, monitoring service 6 operates on one or more networking protocols such that when IMD 16 communicates protocol messages requesting data and/or application services, networking infrastructure directs those messages to a network address/location of the at least one remote computing device (e.g., server).

Processing circuitry of medical system 10, e.g., IMD 16, external device 24, one or more remote computing devices of monitoring service 6, and/or of one or more other computing devices, may be configured to perform the example techniques of this disclosure. Processing circuitry of IMD 16 may be communicably coupled to one or more sensors (e.g., one or more accelerometers), each being configured to sense patient physiological activity (e.g., physiological parameters) in some form, and sensing circuitry configured to generate sensor data and other patient data. IMD 16 includes a plurality of electrodes (not shown in FIG. 1), and is configured to sense patient cardiac activity in some form (e.g., cardiac electrical activity) via the plurality of electrodes. Processing circuitry of medical system 10, such as processing circuitry of IMD 16, processing circuitry of the one or more remote computing devices of monitoring service 6, and/or processing circuitry of external device 24, may compute values representing some aspect of patient 14's physiology at a particular point-in-time and the computation of these values may be in accordance with a number of applicable metrics and other mechanisms for computing such patient data. As described herein, processing circuitry of IMD 16, possibly in combination with processing circuitry of external device 24, may be operative to capture the above patient data over a period of time and then, analyze the captured physiological parameter values for indicia of patient health including non-trivial changes in patient health. In some examples, the processing circuitry of medical system 10 analyzes patient cardiac activity data (e.g., a cardiac EGM or ECG) and other patient physiological activities sensed by IMD 16 and may identify indicia of a cardiac episode, such as an arrhythmia, or another cardiac event that either has occurred or is occurring in patient 14.

The present disclosure describes a number of example techniques where (e.g., at least one device of) medical system 10 calibrates one or more configurable settings of a medical device to accurately generate a determination regarding some aspect of patient health. In some examples of medical system 10, the medical device being calibrated is operative to analyze patient data including data encoded in one or more physiological signals representing various patient activities (e.g., cardiac activities) sensed by various sensors and may identify indicia of the patient's health in general or for specific aspect of the patient's health. Processing circuitry of IMD 16, as one example medical device, may employ the various sensors to capture such physiological signals over a period of time and then, analyze (e.g., physiological parameter values encoded in) the captured signals for non-trivial changes in patient 14's cardiac health, such as a cardiac malady or abnormality (e.g., asynchrony). The present disclosure describes IMD 16 as having access to a variety of hardware/software devices (e.g., as sensors coupled to IMD 16 and/or components of IMD 16) for generating the above signals, including electrodes, accelerometers, pressure sensors, force transducers, among other sensors.

Monitoring service 6 may be beneficial to patients and clinicians (e.g., caregivers) in any environment. IMD 16 may provide medical care to patient 14 while in clinical setting (e.g., an office) and the above determination of IMD 16 may be presented to patient 14's clinician. Monitoring service 6 may coordinate IMD 16, external device 24 and other device in completing one or more clinical workflow actions. An example workflow may involve a number of tasks, including an evaluation of the patient cardiac activity data, a determination as to whether a cardiac episode occurred, and/or a submission of a service request to monitoring service 6. Another task may be updating the current/default settings when new settings information is returned in response to the service request.

Monitoring service 6 may define rules for a rules-based engine (or other mathematical model) and/or components (e.g., algorithms) of a machine learning model. Monitoring service 6 may be configured to feed, into the rules-based engine and/or the machine learning model, input data identifying setting(s) for IMD 16, and then, generate, for communication to patient 14, output data indicating parameter values and other settings information (e.g., arrhythmia detection criteria) for modifying the device setting(s) and improving IMD 16 operation.

In general, IMD 16 is operative to monitor patient data for cardiac episodes and includes detection logic to effectuate the cardiac monitoring operation(s). The detection logic (e.g., a rules-based engine and/or a machine learning model) may implement the current/default settings by configuring itself using data such as the parameter values and other settings information. Initially, default/previous settings information may be programmed into the detection logic and with those settings, IMD 16 may achieve a certain level of performance; because the modified setting(s) are conducive to patient 14's physiology and/or IMD 16's device type, implementing the modified setting(s), IMD 16 should improve that level of performance and, in some examples, calibrate (e.g., personalize) the detection logic to patient 14. In some examples, IMD 16 may be calibrated for similar patients to patient 14 including patients who are in a same demographic or with a similar condition. This can be accomplished by monitoring service 6 directing the building of the rules-based engine and/or the machine learning model to data from only those similar patients and patient 14 instead of the entire patient population. This may result in the best possible performance of IMD 16 for that patient group. In another example, monitoring service 6 may build the rules-based engine and/or the machine learning model only using data for patient 14, resulting in the best possible performance (e.g., for the reason for monitoring).

IMD 16 may generate a user interface (UI) to display the modified device setting(s) and, in some examples, receive user input via one or more input devices. Receiving the output data may trigger an update to the detection logic of IMD 16, for example, by (re)programming the detection logic with the modified setting(s). IMD 16 receives the output data and updates corresponding parameters and other settings. In this manner, the parameter values and the other settings information from monitoring service 6 replace at least a portion of the current/default settings of IMD 16. Implementing the modified setting(s) enhances the predictive performance of IMD 16 with an improvement in accuracy. The modified setting(s) may cause a reduction in false determinations (e.g., false positives or false negatives) and/or an increase in true determinations (e.g., true negatives or true positives). In some examples, the modified setting(s) results in 1 MB 16 detecting at least one more true cardiac episode than with current/default settings.

The UI may include functionality for enabling UI components (e.g., buttons) and input devices (e.g., a keyboard) through which the clinician may specify their selection of the default/current setting(s) through the UI to 1 MB 16 respective parameter values. IMD 16 may configure the UI to handle each clinician selection by determining which parameters are used in the selected setting(s) and then, identifying respective values for the determined parameters and any other settings information. Similar patients in the same patient group as patient 14 may have the same/compatible parameter values programmed into their medical devices and therefore, may achieve a same performance level.

As described herein, monitoring service 6 is configured to connect with IMD 16 via a wireless communication link and (e.g., automatically) configure the cardiac monitoring/detection functionality of 1 MB 16 to perform at a higher level. IMD 16 may have implemented a number of configurable settings for controlling the cardiac monitoring/detection functionality. In some examples, IMD 16—which may be initially configured with default settings or previously configured with current settings— communicates a request for an automatic configuration and, in response, monitoring service 6 provides IMD 16 with modified settings most likely to result in accurate determination of cardiac episodes. When given example patient cardiac activity data to evaluate for at least one suspected cardiac episode, monitoring service 6 may return the modified settings that are at least more likely than the default settings or the current settings to accurately detect true episodes. In some examples, IMD 16 may be active and in service on patient 14 for at least some time before initiating reconfiguration of its current settings. In some examples, monitoring service 6 configures IMD 16 with settings that are calibrated for devices of a same device type (e.g., of a same model output class) as IMD 16 and based on those devices' shared characteristics (e.g., features) of IMD 16, including device type, device manufacturer, and/or the like. In other examples, monitoring service 6 configures IMD 16 with calibrated settings for characteristics (e.g., features) of patient 14 such that IMD 16 is capable of accurately determining whether patient 14's cardiac activity is indicative of a cardiac episode; in such a case, IMD 16 may not be applicable to other patients, especially those unlike patient 14 with respect to personal cardiac activity. There are a number of possible characteristics to group patient 14 with similar patients of which some examples include patient age or age group, patient condition, patient gender, and any other patient demographic.

Another possible characteristic for calibrating the settings of IMD 16 for patient 14 may be clinician input, such as the clinician's reason for monitoring patient 14, a clinician's annotation(s) for the patient cardiac activity data, or any other clinician setting. Monitoring service 6 may sort into groups a patient population according to the clinician's reason for monitoring, resulting in settings information for at least one group of patients that underwent an evaluations for the same particular reason for monitoring setting. As described herein, for each patient 14 in same group, IMD 16 (or a similar device) performs operations for the evaluation of (e.g., a recorded sampled of) patient cardiac activity for cardiac episodes (e.g., an arrhythmia detection). IMD 16 may be configured to make the configurable settings available for activation. The clinician may manually select any one or more of the following example settings: the reason for monitoring, the patient age, and/or the like.

As an alternative, the clinician selection of one or more settings may be expressed as an annotation to a cardiac EGM (e.g., sample). The annotation may denote additional information from the clinician including an initial arrhythmia determination and any other settings information. The clinician, IMD 16, or both the clinician and IMD 16 may provide the initial determination for the cardiac EGM. After recording the cardiac EGM, the clinician may use IMD 16 to annotate a digital copy of the cardiac EGM by coupling an annotation that reflects the corresponding settings information in effect for the initial determination. The clinician may further couple the annotated cardiac EGM to a service request for monitoring service 6 to return more appropriate settings information for IMD 16 and/or patient 14 or others in the same group.

Amongst the examples of the configurable settings (e.g., reason for monitoring, current date/time, patient date of birth, device type, and/or patient characteristic), IMD 16 may implement default (e.g., pre-determined) settings information for each setting. IMD 16 may be configured with functionality and respective settings information for each of the following example reasons for monitoring: Syncope, Cryptogenic Stroke, Suspected AF, AF Ablation, AF Management, Palpitations, Ventricular Tachycardia, Seizures, Tachy Detection Interval, and/or the like. IMD 16 may define a variety of parameters for effectuating the above example reasons for monitoring of which some example parameters include a monitoring information parameter, a sensing parameter, a demographics parameter, a device history (e.g., episode counter) parameter, and/or the like. For each configurable setting, IMD 16 may execute an algorithm incorporating one or more of the above parameters and one or more other settings information. Examples of other settings information include detection criteria for different types of arrhythmias; for each set of detection criteria, an example parameter may indicate whether a given setting includes an evaluation of the arrhythmia type for that detection criteria. It should be noted that the are substantial number of known and unknown parameters that can be programmed into IMD 16. Some parameters may be required while some parameters may be optional when implementing a particular device setting.

The clinician may suspect patient 14 of having the underlying condition (e.g., arrhythmia) for the selected setting(s) including a reason for monitoring and use IMD 16 to confirm or reject that suspicion. Once the reason for monitoring is selected, IMD 16 may set corresponding parameters to values (e.g., default values or reprogrammed values) reflected in local memory and commence operation. IMD 16 may be configured to sense cardiac activity for patient 14 and then, evaluate that cardiac activity using the algorithm for the selected setting(s).

One example of the monitoring information parameter includes a mobile application optimization parameter. This parameter refers to a mobile application optimization feature of a mobile device operating system, and its values indicate that this feature is either disabled or enabled. In some examples, when mobile application optimization is enabled, patient 14 may experience faster connectivity with IMD 16 (e.g., when creating a service request for monitoring service 6 and/or marking a symptom) via a patient application running on patient device 24; however, this parameter may negatively affect the battery longevity of IMD 16. As described herein, some patients 14 benefit from enabling mobile application optimization while other patients require battery longevity on their medical devices. For the other patients, monitoring service 6 may have disabled the mobile application optimization parameter to calibrate current/default settings of their medical devices, and for patient 14 and any similar patient of same group, vice versa. In some examples, the clinician may set the mobile application optimization parameter to enabled/disabled via a clinician mobile application, an Internet or web application, or a user interface to IMD 16. Monitoring service 6 and/or IMD 16 may benefit patient 14 by enabling the mobile application optimization parameter in the current/default settings information for each reason for monitoring. Thus, the clinician selecting a particular reason for monitoring patient 14 may result in enabling the mobile application optimization parameter and the other calibrated parameter(s) for IMD 16.

Demographic parameters may identify a demographic classification for patient 14. First name, last name, date of birth, gender, phone number, address, etc. are among the example demographics parameters.

Device history parameters may include episode counters, each of which tracks a total number of (true) detected episodes in respective episode types. The episode counters are maintained for the lifetime of IMD 16 or any portion thereof. Examples of episode counters for detections of symptom (e.g., patient-activated) episodes, Tachy episodes, Pause episodes, Brady episodes, AT episodes, AF episodes, and PVCs (% beats) episodes). Other types of counters are possible device history parameters; as one example parameter, an episode timer may track a duration of a given AT/AF episode.

Sensitivity is a metric for evaluating IMD 16 performance and an example sensing parameter. The sensitivity parameter that defines a minimum threshold for R-wave sensing such that only signals that are higher than the sensing threshold are sensed as R-waves. The sensing threshold may define a minimum electrical amplitude that is recognized as a sensed ventricular event. Programming the sensitivity to a higher setting decreases the number of sensed cardiac episodes (e.g., ventricular events with lower amplitudes). Programming the sensitivity to a lower setting increases the number of sensed cardiac episodes (ventricular events), but may result in the oversensing of EMI, myopotentials, P-waves, and T-waves.

Other example sensing parameters include Blank after Sense and T-wave blanking interval. Blank after Sense is a parameter whose value determines a length of the Blank after Sense interval, which starts after the detection of a sensed R-wave. During the Blank after Sense interval, sensing is inhibited to prevent the multiple sensing of the R-wave due to a broad QRS complex. If the Blank after Sense interval parameter is programmed too long, Tachy events may be blanked. T-wave Blanking Interval is a parameter whose value selects a length of the interval during which the sensing threshold remains at its initial value after the detection of an R-wave. To ensure proper sensing of R-waves, monitoring service 6 may create a rule or a model component (e.g., criterion) for when programming these parameters, the T-wave Blanking Interval is equal to or longer than the Blank after Sense interval. If the Blank after Sense interval is programmed to be an interval longer than the T-wave Blanking Interval, the T-wave Blanking Interval will be made equal to the Blank after Sense interval. If IMD 16 includes detection logic configured to place a VS marker under the T-wave (commonly called T-wave oversensing) and monitoring service 6 identifies the VS marker, monitoring service 6 configures an applicable rule or ML component that, in response to VS marker, sets an extended T-wave Blanking Interval that may overcome the issue.

Monitoring service 6 may provide modified settings information from data associated with patient 14's physiology or a similar physiology in order to maximize the accuracy of IMD 16 with respect to cardiac episode detection. In some examples, monitoring service 6 receives a dataset comprising demographic parameters for patient 14, a device type of IMD 16, a clinician's reason for monitoring (e.g., Suspected AF), and determines an output dataset comprising modified settings information including detection criteria for a particular arrhythmia type. Monitoring service 6 may generate the modified settings information to adjust the criteria for detecting arrhythmia episodes and the sensing parameters to improve R-wave sensing. In the modified settings information, monitoring service 6 may recommend programming the sensitivity parameter to a setting slightly above the P-wave amplitude.

If necessary, the clinician may use a mobile application or IMD 16 to adjust the criteria for detecting arrhythmia episodes and other parameters and based on those adjustments, IMD 16 may improve in accuracy. The clinician may check the ECG/EGM trace for the effect of the reprogrammed/programmed settings. Those adjustments are propagated to monitoring service 6 where the accuracy improvement further trains the rules-engine or the machine learning model to predict more accurate parameter values for the configurable device settings. The clinician may use a surface trace with marker annotations in a cardiac EGM window to assess ventricular sensing and possibly, detect undersensing when distinct R-waves are not being marked as ventricular senses (VS) in the Marker Channel. The clinician may indicate possible oversensing by checking the Marker Channel for sensed ventricular events that are not due to sensed R-waves. Each annotation provides monitoring service 6 with training data to fine-tune the current parameter values, further calibrating the detection logic of IMD 16 for monitoring patient 14.

The clinician may also suspect that transient R-wave amplitudes are the cause of a loss of sensing and program a more sensitive value, while still ensuring that the sensitivity parameter value is greater than the amplitude of the patient's P-waves. This adjustment also may be used to further train the rules or module components (e.g., algorithms) in determining the setting information most likely to result in a performance improvement (e.g., maximizing accuracy). Monitoring service 6 may store the more sensitive parameter value and use that value to modify settings of other devices and/or other patients (e.g., a same patient group). Monitoring service 6 may further adjust the sensitivity parameter based on additional training and then, distribute this adjustment, for example, further calibrating the detection logic of IMD 16 for monitoring patient 14.

Monitoring service 6 provides modified setting information to reprogram IMD 16 to suit patient 14 as an individual patient. The modified setting information may adjust the criteria by which an increased ventricular rhythm is classified as a Tachy episode. As one example criterion, interval, may select the ventricular interval length of the rate that will be classified as ventricular tachyarrhythmia. Another example criterion, duration, may select the number of Tachy events that must occur before the episode is classified as a Tachy episode. The tachy criteria may include other parameters and/or detection criteria.

Certain patients may respond better than other patients to evaluations by IMD 16 for a particular device setting (e.g., reason for monitoring). IMD 16 may be configured with detection logic having a tendency to determine episodes that monitoring service 6 rejects as false positive. Patient 14 may, in particular, have a number of false positive episodes. Monitoring service 6 may be use first model 7 or second model 8 to modify information defining the particular device setting and, for example, reduce the number of false positives for patient 14.

In one example, monitoring service 6 provides calibrated/personalized Ectopy Rejection parameter values to prevent false determinations by IMD 16. Several parameters may be arranged (e.g., into an algorithm) for a configurable setting of IMD 16, and based on other parameters, IMD 16 may provisionally (yet incorrectly) detect a true episode (e.g., an AF episode) and then, invoke corresponding criteria (e.g., an algorithm) for the Ectopy Rejection to block the true episode detection. Instead of detecting the true episode, IMD 16 may generate output data indicating that no episode has been detected and/or a false positive cardiac episode.

IMD 16 may reject an AF episode as a false positive when corresponding detection criteria identifies evidence of ectopy during any 2 min period. AF detection criteria may be configured to detects regular and irregular atrial arrhythmias. This feature is appropriate of patient 14 is experiencing palpitations and rapid heartbeats associated with atrial arrhythmias. The AF detection criteria facilitates monitoring for the recurrence of atrial arrhythmias in patients who are post atrial ablation as well as monitoring AT/AF burden and the occurrence of asymptomatic episodes of AT/AF. The AF detection criteria may assess whether medical treatment is necessary or should be adjusted.

The primary cause of a false positive AF detection is runs of ectopy (PACs or PVCs) with irregular coupling intervals caused by underlying sinus variability. In the detection logic, the Ectopy Rejection criteria (e.g., algorithm) recognizes patterns of ectopy by the density of points, for example, in a Lorenz Plot. Additionally, a P-wave presence criteria looks for evidence of a P-wave between two R-waves. IMD 16 may set the Ectopy Rejection parameter to Nominal and enable the P-wave presence criteria. IMD 16 may set the Ectopy Rejection to Aggressive and enable both the P-wave presence criteria and the Ectopy Rejection criteria. When Ectopy Rejection is set to Off, both the P-wave presence criteria and the Ectopy Rejection criteria are disabled.

As illustrated in FIG. 1, monitoring service 6 includes a number of models 8 (e.g., machine learning models, rules-engines, or mathematical models) for use in completing service-related operations. To illustrate by way of example, monitoring service 6 applies first model 7 to an input dataset comprising the patient cardiac activity data and current or default configurable settings that are programmed into detection logic of IMD 16 or another medical device. As described herein, the detection logic is configured to determine whether sensed physiological activity is indicative of a cardiac episode for a patient. Monitoring service 6 may be configured to determine whether modified values of the configurable settings, when implemented by the detection logic, would change an initial detection, by the medical device, of the cardiac episode in the sensed physiological activity. Monitoring service 6 may use the modified values to update the detection logic and then, test that updated detection logic to see if there is any change to the initial detection. In response to a determination that the modified settings would change the initial detection of the cardiac episode for the patient, monitoring service 6 may generate output data indicative of a rejection of the initial detection. On the other hand, in response to a determination that the modified settings would not change the initial detection of the cardiac episode based on the default or current values of the configurable settings, monitoring service 6 may generate output data indicative of a confirmation of the initial detection.

As described herein, IMD 16 may submit patient cardiac activity data suspected to be a cardiac episode (i.e., a determination) and in turn, monitoring service 6 applies first model 7 to the patient cardiac activity data to determine whether modified values for the IMD 16's configurable settings would most likely result in a change to the determination (e.g., a more accurate determination) regarding the patient cardiac activity data (e.g., a false positive determination or a different suspected cardiac episode). It should be noted that IMD 16 may submit, as an alternative determination, patient cardiac activity data that is not suspected to be a cardiac episode of any type, and in turn, monitoring service 6 applies first model 7 to determine whether modified values for the configurable settings would result in a suspected cardiac episode (e.g., a false negative determination).

First model 7 may be structured in a ML architecture comprising a plurality of components in which a component may also be a ML model. In some examples, first model 7 may comprise a first component to confirm an initial determination of a cardiac episode (e.g., as true) or reject that initial determination (e.g., as false/incorrect). Output data from first model 7 may determine whether a second component is applied to patient cardiac activity. If the first component generates output data indicating that the initial determination is true, first model 7 (e.g., and the detection logic) may skip/omit the second component in the evaluation of patient cardiac activity. If the first component generates output data indicating that the initial determination is false, first model 7 (e.g., and the detection logic) feeds the patient cardiac activity to the second component and invokes an associated algorithm. In some examples, the second component is configured to determine whether modified values for the IMD 16's configurable settings would most likely result in a different cardiac arrhythmia type or a false positive. The modifies values may result in as a more accurate determination.

Monitoring service 6 may be configured to remove a flag of true episode from the above initial detection of the cardiac episode by IMD 16. Monitoring service 6 may be configured to determine an annotation or label indicative of a probability score for the initial detection of the cardiac episode. Monitoring service 6 may be configured to modify any diagnostic/metric used in the computing the probability score and/or the initial detection of the true episode, such as daily AF burden. Monitoring service 6 (e.g., via external device 24) may be configured to modify AF detection device programming using remote programming capabilities in IMD 16 to improve detection performance. Monitoring service 6 may be configured to apply a physician or clinician annotation of the true episode after the initial detection episode classification and then, couple that annotation with an episode classifier adjudication to either modify device programming or further improve the detection logic in IMD 16.

IMD 16 may be automatically configured with the modified settings or other settings while, in other examples, IMD 16 may be automatically configured with calibrated settings without having to submit any detection results. A clinician may use IMD 16 to submit a service request to have its current/default settings calibrated for patient 14's personal medical care. This may occur after the clinician annotates Monitoring service 6, as an option, may run in an automated manner such that when IMD 16 is brought online to medical system 10, monitoring service 6 applies second model 9 to IMD 16's features (e.g., device type or another model class) and generates the calibrated settings for IMD 16 (e.g., appropriate settings for patient 14's physiology). Logical components known as micro-services perform these service-related operations as described in further details for FIG. 6. Example micro-services perform respective processes for training and evaluating one or more of models 8.

The present disclosure describes a number of example techniques where (e.g., at least one device of) medical system 10 calibrates one or more configurable settings of a medical device to accurately generate a determination regarding some aspect of patient health. In some examples of medical system 10, IMD 16 is to analyze patient data including data encoded in one or more physiological parameter signals representing various patient activities (e.g., cardiac activities) sensed by one or more sensors and, perhaps, generate analysis results identifying indicia of a cardiac malady or abnormality, such as asynchrony.

Depending on the medical device's type, a considerable variety of parameters (e.g., operational parameters, input/output parameters, model parameters including hyperparameters and output class sub-parameters, function call arguments, heart rate thresholds or other patient physiological parameter thresholds, sensing electrode combination, physiological signal sensing thresholds, therapy electrode combination or other therapeutic signal parameters, and/or the like) affect the medical device's performance. The medical device may implement the configurable settings in its logic circuitry and define each device setting with a number of possible selections (e.g., quantities and/or qualities). Monitoring service 6 may implement a machine learning model based on the configurable settings where a number of output classes for a particular setting are based on that setting's possible selections. Monitoring service 6 may use the machine learning model to evaluate a set of values (e.g., parameter values, such as an aggressiveness level for ectopy rejection) for the configurable settings, for example, with respect to the medical device's accuracy in performing its native functionality. In some examples, monitoring service 6 may program, into the medical device's logic circuitry for configuration as one or more device settings, at least one of these parameter values of which a parameter value may be a device-agnostic parameter value or a device-specific parameter value.

In one example, the evaluation by monitoring service 6 may incorporate patient data (e.g., patient physiological data) into the machine learning model (e.g., as one or more features); by doing so, the evaluation may personalize the (predicted) parameter values for the device settings to patient 14. In this manner, personalized settings tailor IMD 16's functionality to the patient's physiology and his/her typical cardiac activity. The detection logic within IMD 16 implements prediction modeling technology, and programming, into its detection logic, values for the personalized settings, facilitates IMD 16 to operate at peak performance—providing the patient with the best possible care. In general, the detection logic implements a model to differentiate normal cardiac activity from cardiac episodes; the personalized values calibrate that logic and its model to the specific cardiac activity of patient 14 such that the model is now configured to differentiate patient 14's normal or healthy cardiac activity from any abnormal and/or dangerous cardiac activity, in effect, enabling IMD 16 to operate at maximum effectiveness.

As an example, IMD 16 may apply the detection logic to a cardiac EGM and identify various indicia of a particular type of a cardiac episode. When calibrated for the patient, IMD 16 may use the detection logic to distinguish the identified indicia that are evidentiary from any that are misleading or false. Certain indicia information may be more informative for patient 14 than other patients, and the model may classify samples or partial samples of cardiac activity as indicative of a true episode or a non-episode. By maximizing the possible intelligence to be gained from the patient 14's cardiac activity data, the calibrated detection logic may achieve a highest accuracy level when identifying evidence for the cardiac episode in that data.

Monitoring service 6 may utilize the wireless communication link to provide IMD 16 with settings information for monitoring cardiac activity for patients in general, for a certain patient population group, or for patient 14 specifically. In some examples, monitoring service 6 may avail another computing system, such as external device 24, to provide IMD 16 with the appropriate settings, such as a local computing system to patient 14. External device 24, under direction of monitoring service 6, may be configured to manage IMD 16's configuration including updates to any of IMD 16's configurable settings, for instance, to maintain a certain level of accuracy.

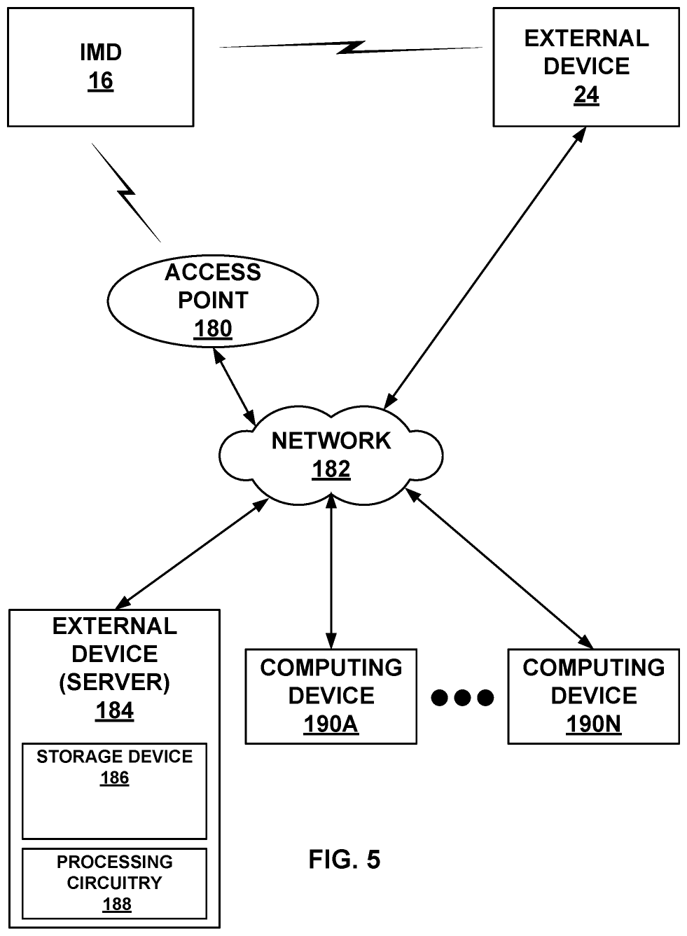

External device 24 may be used to retrieve data (e.g., patient data) from IMD 16. The retrieved data may describe patient 14's cardiac activity for a time period including recorded physiological signals and/or (measured) values of physiological parameters in addition to determinations of (e.g., suspected) episodes of cardiac events such as arrhythmia or other maladies. For example, external device 24 may retrieve cardiac EGM (e.g., segments) recorded by IMD 16 due to IMD 16 determining that an episode of a cardiac arrhythmia or another malady occurred during the segment. As another example, external device 24 may receive data related to the configurable settings described herein for IMD 16. As will be discussed in greater detail below with respect to FIG. 5, one or more remote computing devices may interact with IMD 16 in a manner similar to external device 24, e.g., to program IMD 16 and/or retrieve data from IMD 16, via a network.

Although described in the context of examples in which IMD 16 senses cardiac activity, the present disclosure is directed to example medical devices of example medical system 10 that collect patient data and are equipped with a variety of hardware/software components for performing some functionality with the patient data. Example functionality includes monitoring patient cardiac activity for cardiac episodes. Examples of medical system 10 including one or more external devices or remote computing systems of any type configured to validate a medical device's configurable settings may be configured to implement the techniques of this disclosure.

In example medical system 10, monitoring service 6 may employ external device 24 to configure the settings for IMD 16, e.g., based on user input and/or instruction from monitoring service 6. As described in greater detail below with respect to FIG. 6, a remote computing device may operate monitoring service 6 for IMD 16 via external device 24. External device 24 may communicate service requests from IMD 16 to monitoring service 6 and return the modified settings for the configuration of IMD 16. External device 24 may operate in a manner similar to monitoring service 6, e.g., to program IMD 16 with the modified settings such that IMD 16 achieves a higher level of accuracy.

IMD 16 may include an implantable or wearable monitoring device, a pacemaker/defibrillator, a ventricular assist device (VAD), and other cardiac monitoring devices. IMD 16 may be configured with detection logic to implement one or more of a number of compatible mechanisms configured to successfully monitor patient 14's cardiac activity for evidence of cardiac episodes. The mechanism may be a mathematical model (e.g., rules-based engine) or a machine learning model (e.g., a decision tree), each of which prescribes criterion that the detection logic may use for distinguishing patient data indicative of a true cardiac episode from patient data that does not indicate a true cardiac episode. Medical device manufacturers and/or medical device software developers may produce different versions of the detection logic where some versions perform at different accuracy levels.

As described herein, other factors may influence performance of even the same IMD 16 version. These factors include features corresponding to patient 14 (e.g., patient population or population sub-group). By grouping together patients having a same medical condition or reason for monitoring, monitoring service 6 may determine settings information designed for cardiac episode detection given the same condition or reason for monitoring. The determined settings information allows IMD 16 to distinguish true episodes from episodes that are false but under a prior settings configuration, detected as “true” episodes. Patients of a patient population or a sub-population may be grouped according to similar demographic parameters such that patient 14 may be in a group of patient of a same age, gender, marital status, class, and/or the like. Other factors include features corresponding to the environment of patient 14 and/or version of IMD 16.

Monitoring service 6 may employ data analytics (e.g., Medtronic Cardiac Compass®) to programming and interrogation events, such as when a patient's device is remotely interrogated or remotely programmed. Some analytics generate trend graphs reporting time stamps when IMD 16 was interrogated or reprogrammed to allow possible correlations between parameter changes and other clinical trends. Monitoring service 6 may incorporate the trend graphs into first model 7 or second model 8 (e.g., as a neural network layer) and possibly to account for the correlations, adjust current settings information of IMD 16. Assuming the adjustment results in improved performance, the adjusted settings information is retained until a next update is triggered. However, if the adjustment causes performance loss, the adjusted settings information may be further adjusted to correct such a loss. IMD 16 and/or monitoring service 6 may revert adjusted parameter value(s) back to a previous (e.g., default) value or modify the adjusted parameter value(s) to a different value with an expected performance gain mitigating the performance loss.

With respect to the above-mentioned detection logic, current or default settings of IMD 16 (or another medical device in medical system 10) may correspond to programmable parameters of the detection logic. In some examples, IMD 16 is configured with machine learning resources (e.g., machine learning structures and functionality) such as a model configured to detect a cardiac episode from patient data or otherwise, reject the patient data as a cardiac episode; if the detection logic employed by IMD 16 is to apply such a model, the detection logic may map a specific setting to one or more of the model's parameters (e.g., weights), store, in memory, a value for that specific setting, and then, access that value when the detection logic applies the one or more of the model's parameters to the patient data. As an improvement to medical devices such as IMD 16, the techniques described herein replace data for the current or default settings with modified (e.g., calibrated) settings, resulting in more accurate output classifications by the detection logic.

Monitoring service 6 may maintain one or more databases comprising settings information for a number of classes (e.g., individual medical devices or medical device types, patient populations or patient population sub-groups, and/or the like). An example database may define configurable settings for a specific medical device type (e.g., cardiac monitoring devices or glucose management devices) and/or for a specific patient population (e.g., heart failure patients or diabetes patients). Another example database may provide fine-tuned settings information for different versions of an example medical device. For instance, IMD 16 may be an insertable cardiac monitor (ICM) for which the example database specifies parameter data for the configurable settings that have been implemented for ICMs of a same device type. The fine-tuned settings information of the other example database may distinguish parameter values for a same version of ICM from respective parameter values for other ICM versions.

Another example database stores respective settings information corresponding to a plurality of groups (e.g., patient groupings according to demographics such as by age or gender, reasons for monitoring, physiological characteristics, and/or the like) and different parameters may be defined for each group. If monitoring service 6 receives a request identifying a patient by population or population sub-group, monitoring service 6 may query the one or more databases and retrieve information indicating that the patient is in a patient population that responds very well to pacing therapy. Monitoring service 6 may further retrieve information identifying applicable medical devices for the patent population, and based on which medical device submitted the request, monitoring service 6 may return appropriate settings for pacing therapy and, possibly, additional settings; in some examples, the requesting medical device uses the returned appropriate settings to modify its current/default settings. Hence, monitoring service 6 may employ the one or more databases to calibrate/update settings for different medical devices, causing a reduction in clinical as well as optimizing device therapy and diagnostics.

Monitoring service 6 may leverage a machine learning model and/or the one or more databases to determine whether modifying one or more settings causes a change in a determination made by IMD 16 using current/default device settings. To illustrate by way of an example service request, IMD 16 may determine that patient 14's cardiac activity data indicates a suspected cardiac episode of a particular type and then, submit, in the example service request, information identifying such a determination and, possibly, other information such as the cardiac activity data and/or the current/default settings. Based on the example service request, monitoring service 6 may apply the machine learning model and/or query the one or more databases and retrieve appropriate settings information for IMD 16. By comparing the appropriate setting information to the current/default settings, monitoring service 6 may identify one or more settings whose modification should result in improved performance. As an example device setting, a programmable parameter referred to as an ectopy rejection may be set to different levels of aggressiveness such that IMD 16's detection logic may implement a first level of aggressiveness while monitoring service 6 recommends, as a modified setting, a second level of aggressiveness; the first level may be more aggressive than the second level or vice versa. Monitoring service 6 may return the second level of aggressiveness and in turn, IMD 16 may replace the first level of aggressiveness with the second level.

In some examples, before returning to IMD 16 data indicating the second level of aggressiveness as a modified value for the ectopy rejection setting, monitoring service 6 may evaluate patient 14's cardiac activity data based on the second level of aggressiveness and determine whether the cardiac activity data still is indicative of the suspected cardiac episode of the particular type or whether that determination was inaccurate. If implementing the second level of aggressiveness would cause IMD 16 to change its initial determination to one of no cardiac episode or a cardiac episode of another type, monitoring service 6 may return the second level of aggressiveness as a recommended value for the ectopy rejection setting. As an example, the detection logic of IMD 16 may determine that patient 14 has a cardiac episode suspected to be an Atrial Fibrillation (AF) episode but when evaluated by monitoring service 6, this determination may be rejected.

In some examples, the second level of aggressiveness may be appropriate for detecting cardiac episodes in patient 14 because that particular level of aggressiveness is calibrated to (e.g., fit) patient 14's physiology. Monitoring service 6 may determine that patient 14 or other patients of a same patient group respond to the second level of aggressiveness better than the first level of aggressiveness. Even when other settings remain unmodified, a cardiac episode may be more easily detected in patient 14 when the ectopy rejection is set to the second level of aggressiveness. In other examples, the second level of aggressiveness may be calibrated to IMD 16 and the capabilities/capacities of its resources (e.g., hardware/software components). For instance, monitoring service 6 may determine that IMD 16 achieves a higher accuracy rate when the ectopy rejection is set to the second level of aggressiveness instead of another level; furthermore, this determination may hold regardless of which patient is being monitored.

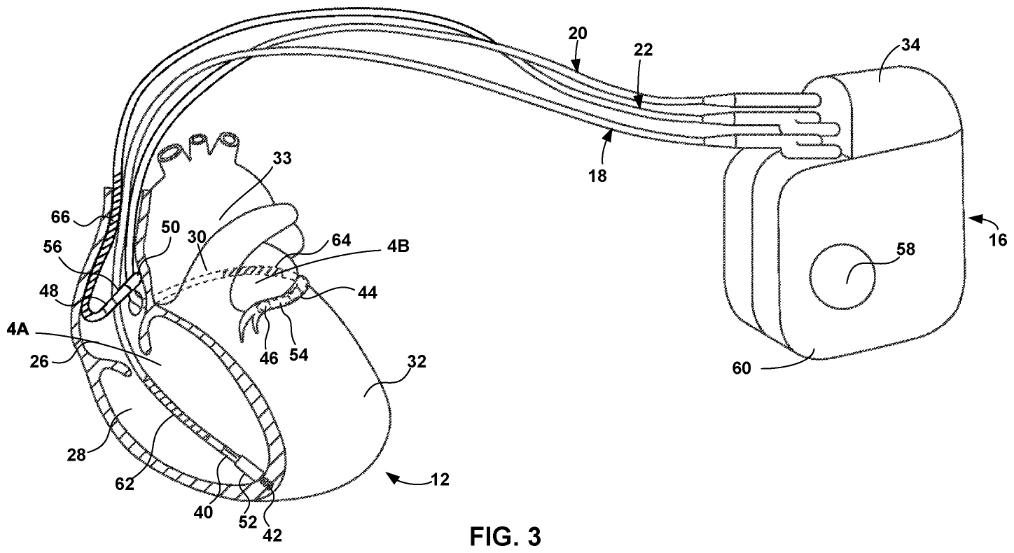

Monitoring service 6 may comprise a micro-service to implement a copy of the detection logic of IMD 16, for example, by applying a third model to features extracted from patient 14's cardiac activity data. As described in detail for FIG. 2, third model 76 represents an example of the above third model. Monitoring service 6 may program the recommended setting values into the detection logic copy, which may update one or more components of the third model including any thresholds, equations, weights, and other logic elements. Based on the application by the micro-service of the updated third model to the extracted features, the detection logic copy may reject the AF determination (e.g., as a false determination under the ectopy rejection setting) and (possibly) determine that the suspected cardiac episode is (more likely) a true episode of one or more Premature Atrial Contractions (PAC). Replacing the first level of aggressiveness with the second level of aggressiveness in the detection logic copy may produce the updated third model, which when compared to the third model at IMD 16, implements one or more different criterion for distinguishing PAC from AF episodes.

Monitoring service 6 may generate this (true) determination in a manner that informs a user of IMD 16, which may be patient 14 or a clinician of patient 14. For example, IMD 16 or external device 24 may run a client application for monitoring service 6 and when recommended settings data is received, the client application may generate a graphical user interface (GUI) element (e.g., a dialog box) to notify patient 14 or the caregiver of the second level of aggressiveness. The GUI element may also include at least one selection mechanism (e.g., a button) that when activated, may invoke a function corresponding to the recommended modified setting, the second level of aggressiveness. In one example, the client application may generate a dialog box with only a drop-down menu and an option to close the dialog box. In another example, the client application may generate a dialog box with separate buttons for accepting and rejecting the recommend modified setting. By activating the button for accepting the recommend setting, the client application may be configured to program the second level of aggressiveness into the detection logic of IMD 16. Otherwise, monitoring service 6 returns a confirmation of IMD 16's initial determination and in turn, the client application running on IMD 16 or external device 24 generates a GUI element for presenting the confirmation on a display device.

In some examples, monitoring service 6 may perform a brute-force method to determine the recommend modified setting and, possibly, one or more additional modified settings. Monitoring service 6 may generate a plurality of sets where each set comprises a unique combination of parameter values, each parameter value of the combination of parameter values corresponding to a respective one of the configurable settings, determine one or more metric values for a baseline corresponding to one of the one of the plurality of sets used in the medical device. For each other one of the plurality of sets, monitoring service determines one or more second metric values corresponding to the combination of parameter values of that set, compares the one or more second metric values to the baseline, and determines whether to change from the one of the plurality of sets corresponding to the baseline to the other one of the plurality of sets based on the comparison.

In some examples, the brute-force method may identify an additional modified settings to recommend. For IMD 16, duration settings are programmable parameters such as a pause duration of Brady number of beats that could be easily adjusted to reduce or eliminate false positives.

FIG. 2 is a conceptual diagram illustrating IMD 16 and leads 18, 20, 22 of medical device system 10 in greater detail. Leads 18, 20, 22 may be electrically coupled to therapy delivery circuitry, sensing circuitry, or other circuitry of IMD 16 via connector block 34. In some examples, proximal ends of leads 18, 20, 22 include electrical contacts that electrically couple to respective electrical contacts within connector block 34. In addition, in some examples, leads 18, 20, 22 are mechanically coupled to connector block 34 with the aid of set screws, connection pins or another suitable mechanical coupling mechanism.

Each of the leads 18, 20, 22 includes an elongated insulative lead body, which may carry a number of conductors separated from one another by tubular insulative sheaths. In the illustrated example, bipolar electrodes 40 and 42 are located proximate to a distal end of lead 18. In addition, bipolar electrodes 44 and 46 are located proximate to a distal end of lead 20 and bipolar electrodes 48 and 50 are located proximate to a distal end of lead 22. Electrodes 40, 44, and 48 may take the form of ring electrodes, and electrodes 42, 46 and 50 may take the form of extendable helix tip electrodes mounted retractably within insulative electrode heads 52, 54 and 56, respectively. Each of the electrodes 40, 42, 44, 46, 48 and 50 may be electrically coupled to a respective one of the conductors within the lead body of its associated lead 18, 20, 22, and thereby coupled to respective ones of the electrical contacts on the proximal end of leads 18, 20 and 22.

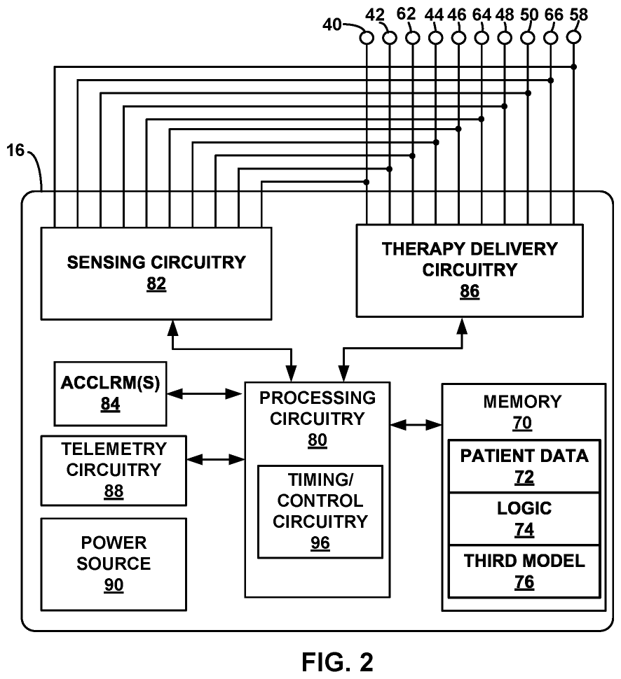

Electrodes 40, 42, 44, 46, 48 and 50 may sense electrical signals attendant to the depolarization and repolarization of heart 12. The electrical signals are conducted to IMD 16 via the respective leads 18, 20, 22. In some examples, 1 MB 16 also delivers pacing pulses to LV 32 via electrodes 44, 46 to cause depolarization of cardiac tissue of heart 12. In some examples, as illustrated in FIG. 2, 1 MB 16 includes one or more housing electrodes, such as housing electrode 58, which may be formed integrally with an outer surface of hermetically-sealed housing 60 of IMD 16 or otherwise coupled to housing 60. In some examples, housing electrode 58 is defined by an uninsulated portion of an outward facing portion of housing 60 of 1 MB 16. Other division between insulated and uninsulated portions of housing 60 may be employed to define two or more housing electrodes. In some examples, housing electrode 58 comprises substantially all of housing 60. Any of the electrodes 40, 42, 44, 46, 48, and 50 may be used for unipolar sensing or stimulation delivery in combination with housing electrode 58. As described in further detail with reference to FIG. 3, housing 60 may enclose therapy delivery circuitry that generates cardiac pacing pulses and defibrillation or cardioversion shocks, as well as sensing circuitry for monitoring the patient's heart rhythm.

In some examples, leads 18, 20, 22 may also include elongated electrodes 62, 64, 66, respectively, which may take the form of a coil. IMD 16 may deliver defibrillation pulses to heart 12 via any combination of elongated electrodes 62, 64, 66, and housing electrode 58. Electrodes 58, 62, 64, 66 may also be used to deliver cardioversion pulses to heart 12. Electrodes 62, 64, 66 may be fabricated from any suitable electrically conductive material, such as, but not limited to, platinum, platinum alloy or other materials known to be usable in implantable defibrillation electrodes.

The configuration of medical system 10 illustrated in FIGS. 1 and 2 is one example, and is not intended to be limiting. In other examples, a therapy system may include extravascular electrodes, such as subcutaneous electrodes, epicardial electrodes, and/or patch electrodes, instead of or in addition to the electrodes of transvenous leads 18, 20, 22 illustrated in FIG. 1. Further, IMD 16 need not be implanted within patient 14. In examples in which IMD 16 is not implanted in patient 14, IMD 16 may deliver defibrillation pulses, pacing pulses, and other therapies to heart 12 via percutaneous leads that extend through the skin of patient 14 to a variety of positions within or outside of heart 12.

In other examples of medical device systems that provide electrical stimulation therapy to heart 12, a therapy system may include any suitable number of leads coupled to IMD 16, and each of the leads may extend to any location within or proximate to heart 12. For example, a therapy system may include a dual chamber device rather than a three-chamber device as shown in FIG. 1. In one example of a dual chamber configuration, IMD 16 is electrically connected to a single lead that includes stimulation and sense electrodes within LV 32 as well as sense and/or stimulation electrodes within RA 26, as shown in FIG. 3. In another example of a dual chamber configuration, IMD 16 is connected to two leads that extend into a respective one of RA 28 and LV 32.

In some examples, a medical device system includes one or more intracardiac pacing devices instead of, or in addition to, an IMD coupled to leads that extend to heart 12, like IMD 16. The intracardiac pacing devices may include therapy delivery and processing circuitry within a housing configured for implantation within one of the chambers of heart 12. In such systems, the plurality of pacing devices, which may include one or more intracardiac pacing devices and/or an IMD coupled to one or more leads, may communicate to coordinate sensing and pacing in various chambers of heart 12 to provide CRT according to the techniques described herein. Processing circuitry and memory of one or more of the pacing devices, and/or another implanted or external medical device, may provide the functionality for controlling delivery of CRT ascribed to processing circuitry and memory of IMD 16 herein.

As illustrated in FIG. 2, IMD 16 is communicatively coupled to one or more sensors that are positioned to sense aspects of a patient's cardiac health. A sensor may be configured in a location (e.g., near heart 12) for sensing cardiac activity, in particular, various physiological parameters corresponding to heart 12. Pressure sensor 2 is configured to generate signals (e.g., physiological parameter signals) to encode pressure data for heart 12 and to avoid negative effects of employing pressure sensor 2 for these signals, IMD 16 uses a model to estimate the same pressure data; if the estimated pressure data satisfies a minimum accuracy requirement, IMD 16 may employ the estimated pressure data in detection logic for monitoring a cardiac abnormality and/or in therapy circuitry to correct the cardiac abnormality.

Although not illustrated in FIG. 2, system 10 may include one or more sensors, e.g., accelerometers or other mechanosensors, operative as intermediate or indirect sensors of pressure data (e.g., left ventricle pressure). As examples, a mechanosensor may be located within IMD 16, or at an endocardial or epicardial location of heart 12, such as locations 4A and 4B, on the right and left ventricular free walls with proximity to the Mitral and Tricuspid valves, or on the left and/or right ventricular apices. The sensors may be disposed on one or more of leads 18, 20, 22, another lead not shown in FIG. 2, or may be wirelessly coupled to IMD 16.

FIG. 3 is a functional block diagram of one example configuration of IMD 16 of FIGS. 1 and 2. In the illustrated example, IMD 16 includes memory 70, processing circuitry 80, sensing circuitry 82, one or more accelerometers 84, therapy delivery circuitry 86, telemetry circuitry 88, and power source 90, one or more of which may be disposed within housing 60 of IMD 16.

In some examples, memory 70 includes computer-readable instructions that, when executed by processing circuitry 80, cause IMD 16 and processing circuitry 80 to perform various functions attributed to IMD 16 and processing circuitry 80 herein. Memory 70 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media. In addition to sensed physiological parameters of patient 14 (e.g., EGM or ECG signals), one or more time intervals for timing fusion pacing therapy and biventricular pacing therapy to heart 12 may be stored by memory 70.

In general, memory 70 may include various information datasets (e.g., database tables) and/or software components (e.g., software programs). As illustrated in the example of FIG. 3, memory 70 may include patient data 72, logic 74, and third model 76. In some examples, third model 76 defines a machine learning model (e.g., a decision tree model or a neural network) that is implemented by logic 74 (e.g., detection logic) when applied to patient data 72.

Processing circuitry 80 may include fixed function circuitry and/or programmable circuitry. Processing circuitry 80 may include one or more of a microprocessor, a controller, digital signal processing circuitry (DSP), an application specific integrated circuit (ASIC), a field-programmable gate array (FPGA), or equivalent discrete or integrated logic circuitry. In some examples, processing circuitry 80 may include multiple components, such as any combination of one or more microprocessors, one or more controllers, one or more DSPs, one or more ASICs, or one or more FPGAs, as well as other discrete or integrated logic circuitry. The functions attributed to processing circuitry 80 herein may be embodied as software, firmware, hardware or any combination thereof.

Processing circuitry 80 may be configured to determine a heart rate of heart 12 based on electrical activity sensed by sensing circuitry 82, estimate left ventricle pressure data (e.g., LV pressure and/or a derivative thereof) from leveraging machine learning resources of model data 76 of memory 70 and from various sensor data memorialized in patient data 72 (e.g., which may be codified into structured datasets of physiological parameters), and control the delivery CRT to heart 12 by therapy delivery circuitry 86 based on the left ventricular pressure data. With further respect to this example, processing circuitry 80 may be configured to cause therapy delivery circuitry 86 to deliver electrical pulses.

Sensing circuitry 82 is configured to monitor signals from at least one of electrodes 40, 42, 44, 46, 48, 50, 58, 62, 64 or 66 in order to monitor electrical activity of heart 12, e.g., via EGM signals. For example, sensing circuitry 82 may sense atrial events (e.g., a P-wave) with electrodes 48, 50, 66 within RA 26 or sense an LV 32 event (e.g., an R-wave) with electrodes 44, 46, 64 within LV 32. In some examples, sensing circuitry 82 includes switching circuitry to select which of the available electrodes are used to sense the electrical activity of heart 12. For example, processing circuitry 80 may select the electrodes that function as sense electrodes via the switching circuitry within sensing circuitry 82, e.g., by providing signals via a data/address bus. In some examples, sensing circuitry 82 includes one or more sensing channels, each of which may comprise an amplifier. In response to the signals from processing circuitry 80, the switching circuitry of sensing circuitry 82 may couple the outputs from the selected electrodes to one of the sensing channels.

In some examples, one channel of sensing circuitry 82 may include an R-wave amplifier that receives signals from electrodes 40 and 42, which are used for pacing and sensing in RV 28 of heart 12. Another channel may include another R-wave amplifier that receives signals from electrodes 44 and 46, which are used for pacing and sensing proximate to LV 32 of heart 12. In some examples, the R-wave amplifiers may take the form of an automatic gain controlled amplifier that provides an adjustable sensing threshold as a function of the measured R-wave amplitude of the heart rhythm.

In addition, in some examples, one channel of sensing circuitry 82 may include a P-wave amplifier that receives signals from electrodes 48 and 50, which are used for pacing and sensing in RA 26 of heart 12. In some examples, the P-wave amplifier may take the form of an automatic gain controlled amplifier that provides an adjustable sensing threshold as a function of the measured P-wave amplitude of the heart rhythm. Examples of R-wave and P-wave amplifiers are described in U.S. Pat. No. 5,117,824 to Keimel et al., which issued on Jun. 2, 1992 and is entitled, “APPARATUS FOR MONITORING ELECTRICAL PHYSIOLOGIC SIGNALS,” and is incorporated herein by reference in its entirety. Other amplifiers may also be used. Furthermore, in some examples, one or more of the sensing channels of sensing circuitry 82 may be selectively coupled to housing electrode 58, or elongated electrodes 62, 64, or 66, with or instead of one or more of electrodes 40, 42, 44, 46, 48 or 50, e.g., for unipolar sensing of R-waves or P-waves in any of chambers 26, 28, or 32 of heart 12.

In some examples, sensing circuitry 82 includes a channel that comprises an amplifier with a relatively wider pass band than the R-wave or P-wave amplifiers. Signals from the selected sensing electrodes that are selected for coupling to this wide-band amplifier may be provided to a multiplexer, and thereafter converted to multi-bit digital signals by an analog-to-digital converter for storage in memory 70 as an EGM. In some examples, the storage of such EGMs in memory 70 may be under the control of a direct memory access circuit. Processing circuitry 80 may employ digital signal analysis techniques to characterize the digitized signals stored in memory 70 to detect and classify the patient's heart rhythm from the electrical signals. Processing circuitry 80 may detect and classify the heart rhythm of patient 14 by employing any of the numerous signal processing methodologies known in the art.

Signals generated by sensing circuitry 82 may include, for example: an RA-event signal, which indicates a detection of a P-wave via electrodes implanted within RA 26 (FIG. 1); an LA-event signal, which indicates a detection of a P-wave via electrodes implanted within LA 33 (FIG. 1); an RV-event signal, which indicates a detection of an R-wave via electrodes implanted within RV 28; or an LV-event signal, which indicates a detection of an R-wave via electrodes implanted within LV 32. In the example of medical system 10 shown in FIGS. 1 and 2, IMD 16 is not connected to electrodes that are implanted within LA 33. However, in other example therapy systems, IMD 16 may be connected to electrodes that are implanted within LA 33 in order to sense electrical activity of LA 33.

In some examples, IMD 16 may include one or more additional sensors, such as accelerometers 84. In some examples, accelerometers 84 may comprise one or more three-axis accelerometers. As described above, accelerometers 84 may be located within a housing of IMD 16, or coupled to IMD by one or more leads or a wireless connection. Signals generated by accelerometers 84 may be indicative of, for example, gross body movement of patient 14, such as a patient posture or activity level, as well as cardiac motion and vibration.

Therapy delivery circuitry 86 is electrically coupled to electrodes 40, 42, 44, 46, 48, 50, 58, 62, 64, and 66, e.g., via conductors of the respective lead 18, 20, 22, or, in the case of housing electrode 58, via an electrical conductor disposed within housing 60 of IMD 16. Therapy delivery circuitry 86 is configured to generate and deliver electrical stimulation therapy. For example, therapy delivery circuitry 86 may deliver a pacing stimulus to LV 32 (FIG. 2) of heart 12, in accordance with the fusion pacing techniques described herein, via at least two electrodes 44, 46 (FIG. 2). As another example, therapy delivery circuitry 86 may deliver a pacing stimulus to RV 28 via at least two electrodes 40, 42 (FIG. 2) and a pacing stimulus to LV 32 via at least two electrodes 44, 46 (FIG. 2), e.g., in accordance with the biventricular pacing techniques described herein.

In some examples, therapy delivery circuitry 86 is configured to deliver cardioversion or defibrillation shocks to heart 12. The pacing stimuli, cardioversion shocks, and defibrillation shocks may be in the form of stimulation pulses. In other examples, therapy delivery circuitry 86 may deliver one or more of these types of stimulation in the form of other signals, such as sine waves, square waves, or other substantially continuous time signals.

Therapy delivery circuitry 86 may include a switching circuitry, and processing circuitry 80 may use the switching circuitry to select, e.g., via a data/address bus, which of the available electrodes are used to deliver defibrillation pulses or pacing pulses. The switching circuitry may include a switch array, switch matrix, multiplexer, or any other type of switching device suitable to selectively couple stimulation energy to selected electrodes. In other examples, processing circuitry 80 may select a subset of electrodes 40, 42, 44, 46, 48, 50, 58, 62, 64, and 66 with which stimulation is delivered to heart 12 without a switching circuitry.

Processing circuitry 80 includes pacer timing and control circuitry 96, which may be embodied as hardware, firmware, software, or any combination thereof. Pacer timing and control circuitry 96 may comprise a dedicated hardware circuit, such as an ASIC, separate from other processing circuitry 80 components, such as a microprocessor, or a software module executed by a component of processing circuitry 80 (e.g., a microprocessor or ASIC). Pacer timing and control circuitry 96 may help control the delivery of pacing pulses to heart 12.

In examples in which IMD 16 delivers a pacing pulse, pacer timing and control circuitry 96 may include a timer for determining that a selected A-V interval has elapsed after processing circuitry 80 determines that an atrial pace or sense event (Apis, or more generally A) has occurred. The timer of pacing timing and control circuitry 96 may be configured to begin upon the detection of the preceding atrial pace or sense event (Apis) by processing circuitry 80. Upon expiration of the particular timer, processing circuitry 80 may control therapy delivery circuitry 86 to deliver a pacing stimulus, according to a fusion or biventricular pacing configuration, to heart 12. For example, pacing timing and control circuitry 96 may generate a trigger signal that triggers the output of a pacing pulse by therapy delivery circuitry 86.

In examples in which IMD 16 is configured to deliver other types of cardiac rhythm therapy in addition to fusion pacing and biventricular pacing, pacer timing and control circuitry 96 may also include programmable counters which control the basic time intervals associated with DDD, VVI, DVI, VDD, AAI, DDI, DDDR, VVIR, DVIR, VDDR, AAIR, DDIR and other modes of single and dual chamber pacing. In the aforementioned pacing modes, “D” may indicate dual chamber, “V” may indicate a ventricle, “I” may indicate inhibited pacing (e.g., no pacing), and “A” may indicate an atrium. The first letter in the pacing mode may indicate the chamber that is paced, the second letter may indicate the chamber in which an electrical signal is sensed, and the third letter may indicate the chamber in which the response to sensing is provided.

In examples in which IMD 16 is configured to deliver other types of cardiac rhythm therapy in addition to CRT, intervals defined by pacer timing and control circuitry 96 within processing circuitry 80 may include atrial and ventricular pacing escape intervals, refractory periods during which sensed P-waves and R-waves are ineffective to restart timing of the escape intervals, and the pulse widths of the pacing pulses. As another example, pacer timing and control circuitry 96 may define a blanking period, and provide signals from sensing circuitry 82 to blank one or more channels, e.g., amplifiers, for a period during and after delivery of electrical stimulation to heart 12. The durations of these intervals may be determined by processing circuitry 80 in response to stored data in memory 70. In some examples, the pacer timing and control circuitry 96 of processing circuitry 80 may also determine the amplitude of the cardiac pacing pulses.

During certain pacing modes, escape interval counters within pacer timing/control circuitry 96 of processing circuitry 80 may be reset upon sensing of R-waves and P-waves. Therapy delivery circuitry 86 may include pacer output circuits that are coupled, e.g., selectively by switching circuitry, to any combination of electrodes 40, 42, 44, 46, 48, 50, 58, 62, or 66 appropriate for delivery of a bipolar or unipolar pacing pulse to one of the chambers of heart 12. Processing circuitry 80 may reset the escape interval counters upon the generation of pacing pulses by therapy delivery circuitry 86, and thereby control the basic timing of cardiac pacing functions, including fusion cardiac resynchronization therapy.

The value of the count present in the escape interval counters when reset by sensed R-waves and P-waves may be used by processing circuitry 80 to measure the durations of R-R intervals, P-P intervals, P-R intervals and R-P intervals, which are measurements that may be stored in memory 70. Processing circuitry 80 may use the count in the interval counters to detect a tachyarrhythmia event, such as ventricular fibrillation event or ventricular tachycardia event. Upon detecting a threshold number of tachyarrhythmia events, processing circuitry 80 may identify the presence of a tachyarrhythmia episode, such as a ventricular fibrillation episode, a ventricular tachycardia episode, or a non-sustained tachycardia (NST) episode. Examples of tachyarrhythmia episodes that may qualify for delivery of responsive therapy include a ventricular fibrillation episode or a ventricular tachyarrhythmia episode.

In some examples, processing circuitry 80 may operate as an interrupt driven device, and is responsive to interrupts from pacer timing and control circuitry 96, where the interrupts may correspond to the occurrences of sensed P-waves and R-waves and the generation of cardiac pacing pulses. Any necessary mathematical calculations to be performed by processing circuitry 80 and any updating of the values or intervals controlled by the pacer timing and control circuitry 96 of processing circuitry 80 may take place following such interrupts. A portion of memory 70 may be configured as a plurality of recirculating buffers, capable of holding series of measured intervals, which may be analyzed by processing circuitry 80 in response to the occurrence of a pace or sense interrupt to determine whether the patient's heart 12 is presently exhibiting atrial or ventricular tachyarrhythmia.

If IMD 16 is configured to generate and deliver defibrillation shocks to heart 12, therapy delivery circuitry 86 may include a high voltage charge circuit and a high voltage output circuit. In the event that processing circuitry 80 determines that generation of a cardioversion or defibrillation shock is required, processing circuitry 80 may employ the escape interval counter to control timing of such cardioversion and defibrillation shocks, as well as associated refractory periods. In response to the detection of atrial or ventricular fibrillation or tachyarrhythmia requiring a cardioversion pulse, processing circuitry 80 may activate a cardioversion/defibrillation control circuitry (not shown), which may, like pacer timing and control circuitry 96, be a hardware component of processing circuitry 80 and/or a firmware or software module executed by one or more hardware components of processing circuitry 80. The cardioversion/defibrillation control circuitry may initiate charging of the high voltage capacitors of the high voltage charge circuit of therapy delivery circuitry 86 under control of a high voltage charging control line.

Processing circuitry 80 may monitor the voltage on the high voltage capacitor, e.g., via a voltage charging and potential (VCAP) line. In response to the voltage on the high voltage capacitor reaching a predetermined value set by processing circuitry 80, processing circuitry 80 may generate a logic signal that terminates charging. Thereafter, timing of the delivery of the defibrillation or cardioversion pulse by therapy delivery circuitry 86 is controlled by a cardioversion/defibrillation control circuitry (not shown) of processing circuitry 80. Following delivery of the fibrillation or tachycardia therapy, processing circuitry 80 may return therapy delivery circuitry 86 to a cardiac pacing function and await the next successive interrupt due to pacing or the occurrence of a sensed atrial or ventricular depolarization.

Therapy delivery circuitry 86 may deliver cardioversion or defibrillation shock with the aid of an output circuit that determines whether a monophasic or biphasic pulse is delivered, whether housing electrode 58 serves as cathode or anode, and which electrodes are involved in delivery of the cardioversion or defibrillation pulses. Such functionality may be provided by one or more switches or a switching circuitry of therapy delivery circuitry 86.

Telemetry circuitry 88 includes any suitable hardware, firmware, software or any combination thereof for communicating with another device, such as external device 24 (FIG. 1). Under the control of processing circuitry 80, telemetry circuitry 88 may receive downlink telemetry from and send uplink telemetry to external device 24 with the aid of an antenna, which may be internal and/or external. Processing circuitry 80 may provide the data to be uplinked to external device 24 and the control signals for the telemetry circuit within telemetry circuitry 88, e.g., via an address/data bus. In some examples, telemetry circuitry 88 may provide received data to processing circuitry 80 via a multiplexer.

In some examples, processing circuitry 80 may transmit atrial and ventricular heart signals (e.g., EGM signals) produced by atrial and ventricular sense amp circuits within sensing circuitry 82 to external device 24. Other types of information may also be transmitted to external device 24, such as the various intervals and delays used to deliver CRT. External device 24 may interrogate IMD 16 to receive the heart signals. Processing circuitry 80 may store heart signals within memory 70, and retrieve stored heart signals from memory 70. Processing circuitry 80 may also generate and store marker codes indicative of different cardiac episodes that sensing circuitry 82 detects, and transmit the marker codes to external device 24. An example pacemaker with marker-channel capability is described in U.S. Pat. No. 4,374,382 to Markowitz, entitled, “MARKER CHANNEL TELEMETRY SYSTEM FOR A MEDICAL DEVICE,” which issued on Feb. 15, 1983 and is incorporated herein by reference in its entirety.

The various components of IMD 16 are coupled to power source 90, which may include a rechargeable or non-rechargeable battery. A non-rechargeable battery may be selected to last for several years, while a rechargeable battery may be inductively charged from an external device, e.g., on a daily or weekly basis. IMD 16 may leverage a remote computing device such as external device 24 and/or remote monitoring service 6 for additional resources (e.g., processing power), for example, to execute a training process for generating a trained version of the machine learning model for implementing the parameter estimation techniques described herein.

Medical system 10 may be a computing system running in a single device or over a network of devices. IMD 16, a medical device, is one example device but other device types may be included. In some examples, an arrhythmia detection method may include any suitable arrhythmia detection algorithm including compatible tachyarrhythmia detection methodologies for processing circuitry 80.

Processing circuitry 80 of IMD 16 may execute logic 74 programmed with model data 76; while under the control of executed logic 74, processing circuitry 80 of IMD 16 applies model data 76 (e.g., as a neural network or a regression function) and part of a computing service to determining which cardiac event type most likely occurred or is occurring.

In some examples, processing circuitry 80, executing logic 74 configured to perform a detection analysis on patient data 72, is operative to detect any change (e.g., a decline) in patient health that may be caused by patient 14's cardiac activity. As part of the detection analysis, processing circuitry 80 may control one or more of sensors to sense, in some form, patient activity. Sensing circuitry 82, as described herein, converts to digital form signals corresponding to the sensed patient activity and provides the digitized signals to processing circuitry 50 for the detection analysis. Sensing circuitry 82 and processing circuitry 80 may store patient data 72 in memory 70 and, in some examples, that stored patient data 72 may include sensor data including activity data from accelerometers 84 and cardiac activity data (e.g., cardiac EGM data or ECG data in the form of one or more graphs on which two-dimensional points and/or vectors are depicted). Various metrics enable the standardized measurement of each sample (e.g., timestamp) of the sensor data and the differentiation between multiple samples (e.g., timestamps or longer time periods and/or patients).

Patient data 72 may store cardiac EGM data encompassing a period of time (e.g., during which a cardiac episode may have occurred in patient 14) and, in some examples, includes a sequence of values representing ECG waves or ECG-type waveforms of a cardiac rhythm. It should be noted that cardiac EGM data for a typical EGM includes a series of samples representing points on waves (e.g., the P wave, Q wave, R wave, S wave, T wave and U wave), intervals (e.g., PR interval, QRS interval (also called QRS duration), QT interval or RR interval), segments (e.g., PR segment, ST segment or TP segment), complex(es) (e.g., QRS complex), and other components. Processing circuitry 50 may apply a pattern recognition technique to interpret waveforms recorded in the cardiac EGM data as one or more of the above EGM components. In some examples, the EGM waveform may indicate an initial detection of a cardiac event/episode. Then, processing circuitry 50 is configured to apply model data 68 to the EGM waveform, and based on prediction values of model data 68, determine whether the cardiac EGM indicates a cardiac episode type. Processing circuitry 50 generates for display output data indicative of a particular cardiac event type as the classification.

Patient data 72 may further include information identifying IMD 16 as a type of medical device, such as by device manufacturer. Patient data 72 may include information identifying detection logic 74 as an algorithm that detects occurrences of cardiac episodes, for instance, by determining whether the cardiac EGM data or ECG data is indicative of such cardiac episodes. Patient data 72 may further include other data corresponding to the patient and/or the sensed patient activity, such as data identifying a patient population sub-group. Patient data 72 may be uploaded to an external device, such as external device 24 of FIG. 1. Sensing circuitry 82 may generate patient data 72 from captured sensor signals that encode the above sensed patient activity including the patient's cardiac activity as described herein.

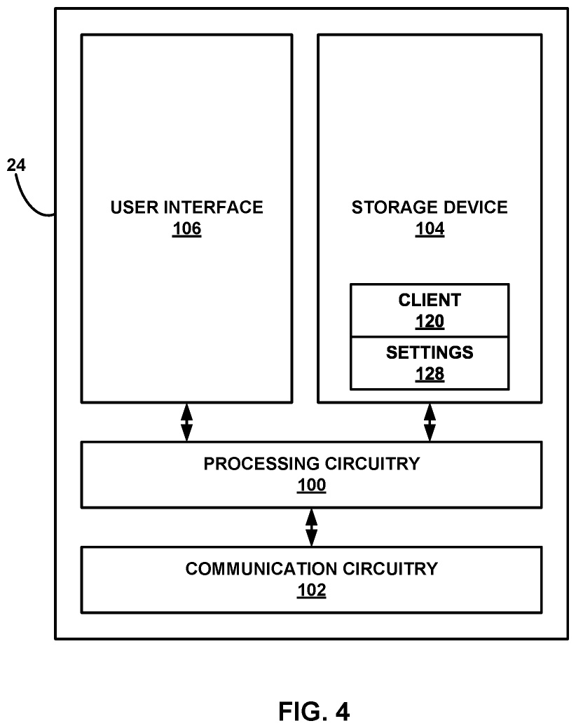

FIG. 4 is a block diagram illustrating an example configuration of components of external device 24. In the example of FIG. 4, external device 24 includes processing circuitry 100, communication circuitry 102, storage device 104, and user interface 106.

Processing circuitry 100 may include one or more processors that are configured to implement functionality and/or process instructions for execution within external device 24. For example, processing circuitry 100 may be capable of processing instructions stored in storage device 104. Processing circuitry 100 may include, for example, microprocessors, DSPs, ASICs, FPGAs, or equivalent discrete or integrated logic circuitry, or a combination of any of the foregoing devices or circuitry. Accordingly, processing circuitry 100 may include any suitable structure, whether in hardware, software, firmware, or any combination thereof, to perform the functions ascribed herein to processing circuitry 100.

Communication circuitry 102 may include any suitable hardware, firmware, software or any combination thereof for communicating with another device, such as IMD 16. Under the control of processing circuitry 100, communication circuitry 102 may receive downlink telemetry from, as well as send uplink telemetry to, IMD 16, or another device. Communication circuitry 102 may be configured to transmit or receive signals via inductive coupling, electromagnetic coupling, NFC, RF communication, Bluetooth, WiFi, or other proprietary or non-proprietary wireless communication schemes. Communication circuitry 102 may also be configured to communicate with devices other than IMD 16 via any of a variety of forms of wired and/or wireless communication and/or network protocols.

Storage device 104 may be configured to store information within external device 24 during operation. Storage device 104 may include a computer-readable storage medium or computer-readable storage device. In some examples, storage device 104 includes one or more of a short-term memory or a long-term memory. Storage device 104 may include, for example, RAM, DRAM, SRAM, magnetic discs, optical discs, flash memories, or forms of EPROM or EEPROM. In some examples, storage device 104 is used to store data indicative of instructions for execution by processing circuitry 100. Storage device 104 may be used by software or applications running on external device 24 to temporarily store information during program execution.

Data exchanged between external device 24 and IMD 16 may include operational parameters. External device 24 may transmit data including computer readable instructions which, when implemented by IMD 16, may control IMD 16 to change one or more operational parameters and/or export collected data. For example, processing circuitry 100 may transmit an instruction to IMD 16 which requests IMD 16 to export collected data (e.g., asystole episode data) to external device 24. In turn, external device 24 may receive the collected data from IMD 16 and store the collected data in storage device 104. The data external device 24 receives from IMD 16 may include patient data (e.g., patient data 72 of FIG. 2), such as data (e.g., cardiac EGMs) corresponding to suspected episodes, patient physiological parameters and other patient activity, sensor data, device manufacturer, software developer.

Processing circuitry 100 may implement any of the techniques described herein to facilitate, on behalf of IMD 16, data and/or application services, for example, from a computing service operating on a remote computing system. Monitoring service 6 is one example computing service, as depicted in FIG. 1, in communication with external device 24. In accordance with one operating mode, monitoring service 6 of FIG. 1 provides values for the configurable settings of IMD 16, for example, when IMD 16 and/or external device 24 establish a network connection and are brought online. When under a different operating mode, monitoring service 6 of FIG. 1 provides values for the configurable settings of IMD 16, for example, in response to a service request from IMD 16 and/or external device 24. In both modes, monitoring service 6 may determine appropriate settings data as described herein for external device 24 to receive and then, communicate to IMD 16. In one example, processing circuitry 100 of external device 24 may run a process that is configured with a connection to a second process (e.g., an agent) running in IMD 16. By invoking functionality of that process, external device 24 may communicate values of configurable settings with instructions directing the second process to replace current or default values of the same settings with the communicated settings data.

As described above, processing circuitry 100 may operate with one or more remote computing devices (e.g., a cloud-based computing system) such as monitoring service 6 as described in further detail with respect to FIG. 6. Processing circuitry 100 may run client 120 which is a client application for monitoring service 6.

In some examples, external device 24, via client 120, coordinates certain operations of monitoring service 6, such as the automatic configuration of a medical device (e.g., IMD 16 of FIG. 1) with appropriate settings. Client 120 may store the appropriate settings in settings 128 and, via a connection with the medical device, direct the medical device on properly implementing settings 128. This may involve a medical device component configured to program into the medical device's detection/determination logic (e.g., logic 74 of FIG. 3). In other examples, client 120 may receive service requests from the medical device for submission to monitoring service 6 and then, proceed to handle those service requests on behalf of the requesting medical device.

A user, such as a clinician or patient 14, may interact with external device 24 (e.g., and client 120) through user interface 106. User interface 86 includes a display (not shown), such as a liquid crystal display (LCD) or a light emitting diode (LED) display or other type of screen, with which processing circuitry 100 may present information related to IMD 16, e.g., episode data including determinations of cardiac episodes based on probability data generated by a machine learning model implemented in logic 74. Other patient data may include sensor data and patient physiological parameters. In addition, user interface 106 may include an input mechanism configured to receive input from the user. The input mechanisms may include, for example, any one or more of buttons, a keypad (e.g., an alphanumeric keypad), a peripheral pointing device, a touch screen, or another input mechanism that allows the user to navigate through user interfaces presented by processing circuitry 100 of external device 24 and provide input. In other examples, user interface 106 also includes audio circuitry for providing audible notifications, instructions, or other sounds to the user, receiving voice commands from the user, or both.

FIG. 5 is a block diagram illustrating an example system that includes an access point 180, a network 182, external computing devices, such as a server 184, and one or more other computing devices 190A-190N (collectively, “computing devices 190”), which may be coupled to IMD 16 and external device 24 via network 182, in accordance with one or more techniques described herein. In this example, IMD 16 may use communication circuitry 54 to communicate with external device 24 via a first wireless connection, and to communicate with an access point 180 via a second wireless connection. In the example of FIG. 5, access point 180, external device 24, server 184, and computing devices 190 are interconnected and may communicate with each other through network 182.

Access point 180 may include a device that connects to network 182 via any of a variety of connections, such as telephone dial-up, digital subscriber line (DSL), or cable modem connections. In other examples, access point 180 may be coupled to network 182 through different forms of connections, including wired or wireless connections. In some examples, access point 180 may be a user device, such as a tablet or smartphone, that may be co-located with the patient. IMD 16 may be configured to transmit data, such as patient data 72, and/or indications of changes in patient health, to access point 180. Access point 180 may then communicate the retrieved data to server 184 via network 182.

In some cases, server 184 may be configured to provide a secure storage site for data that has been collected from IMD 16 and/or external device 24. In some cases, server 184 may assemble data in web pages or other documents for viewing by trained professionals, such as clinicians, via computing devices 99. One or more aspects of the illustrated system of FIG. 5 may be implemented with general network technology and functionality, which may be similar to that provided by the Medtronic CareLink® Network.

In some examples, one or more of computing devices 99 may be a tablet or other smart device located with a clinician, by which the clinician may program, receive alerts from, and/or interrogate IMD 16. For example, the clinician may access data (e.g., patient data) and/or indications of patient health collected by IMD 16 through a computing device 100, such as when patient 14 is in in between clinician visits, to check on a status of a medical condition. In some examples, the clinician may enter instructions for a medical intervention for patient 14 into an application executed by computing device 100, such as based on a status of a patient condition determined by IMD 16, external device 24, server 184, or any combination thereof, or based on other patient data known to the clinician. Device 100 then may transmit the instructions for medical intervention to another of computing devices 190 located with patient 14 or a caregiver of patient 14. For example, such instructions for medical intervention may include an instruction to change a drug dosage, timing, or selection, to schedule a visit with the clinician, or to seek medical attention. In further examples, a computing device 190 may generate an alert to patient 14 based on a status of a medical condition of patient 14, which may enable patient 14 proactively to seek medical attention prior to receiving instructions for a medical intervention. In this manner, patient 14 may be empowered to take action, as needed, to address his or her medical status, which may help improve clinical outcomes for patient 14.

In the example illustrated by FIG. 5, server 184 includes a storage device 186, e.g., to store data retrieved from IMD 16, and processing circuitry 188. Although not illustrated in FIG. 5 computing devices 190 may similarly include a storage device and processing circuitry. Processing circuitry 188 may include one or more processors that are configured to implement functionality and/or process instructions for execution within server 184. For example, processing circuitry 188 may be capable of processing instructions stored in storage device 96. Processing circuitry 188 may include, for example, microprocessors, DSPs, ASICs, FPGAs, or equivalent discrete or integrated logic circuitry, or a combination of any of the foregoing devices or circuitry. Accordingly, processing circuitry 188 may include any suitable structure, whether in hardware, software, firmware, or any combination thereof, to perform the functions ascribed herein to processing circuitry 188. Processing circuitry 188 of server 184 and/or the processing circuitry of computing devices 190 may implement any of the techniques described herein to analyze information received from IMD 16, e.g., to determine whether a determination regarding a patient's cardiac health changes (e.g., from a true arrhythmia detection to a false positive) or to determine appropriate device settings to configure detection logic for cardiac arrhythmias.

Storage device 186 may include a computer-readable storage medium or computer-readable storage device. In some examples, storage device 96 includes one or more of a short-term memory or a long-term memory. Storage device 96 may include, for example, RAM, DRAM, SRAM, magnetic discs, optical discs, flash memories, or forms of EPROM or EEPROM. In some examples, storage device 186 is used to store data indicative of instructions for execution by processing circuitry 188.

FIG. 6 is a block diagram illustrating an example computing service to provide resources to medical devices of an example medical system, such as medical system 10 in FIG. 1, in accordance with one or more examples of the present disclosure. The example computing service, illustrated in FIG. 6 as monitoring service 6, handles service requests from one or more medical devices of the example medical system, such as IMD 16 of FIG. 1. The example computing service may be implemented by the example system of FIG. 5.

In the illustrated example of FIG. 6, computing system 200 refers to a physical or virtualized computing environment in which at least server 210 operates monitoring service 6. Server 210 may be an example of server 184 of FIG. 5 and, in general, includes various hardware/software components that may be configured to run micro-services for monitoring service 6, each micro-service referring to an application or program that constitute at least part of monitoring service 6's functionality. Server 210 may provide a portal (e.g., an Internet portal) through which the one or more medical devices submit service requests. An external device may run a client application for monitoring service 6 and on behalf of the one or more medical devices, may coordinate operations by one or more micro-services running in service 210. One or more servers 211 may support server 210 (e.g., with respect to monitoring service 6's functionality) or may, independently, perform other functionality.

When monitoring service 6 is running, server 210 via communication circuitry 140 receives one or more messages from a medical device such as IMD 16 (or another medical device in medical system 10), and proceeds to handle each message by having processing circuitry 250 of server 210 run an appropriate micro-service according to some examples. As described herein, monitoring service 6 provides the medical device with a number of benefits, for example, by facilitating access to resources (e.g., machine learning resources) and utilization of their functionality.

Processing circuitry 250 of server 210 may employ micro-service(s) for any model used in a computing service. First model 7, second model 9, and/or third model 76 may be configured with a micro-service. First model 7 may have a micro-service operative to expose functionality for submitting requests for determining whether modifying any of IMD 16's configurable settings results in a different determination regarding patient 14's cardiac activity and any suspected cardiac episode in that activity. In a similar manner to the first model's micro-service, micro-service for second model 9 exposes functionality through a user submits requests.

IMD 16's configurable settings may direct the application of the second model in such a manner that modifying one or more settings may affect IMD's performance with respect to detecting cardiac episodes. For example, one or more settings may determine a feature value or a weight to apply to a feature value. Another setting may establish a threshold and/or select certain criteria to for a feature value. Therefore, in order to determine an appropriate configuration for IMD 16, first model micro-service 266 may invoke a micro-service for third model 76 to test potential values for one or more (modified) settings with respect, for example, with respect to accuracy of the suspected cardiac episode.

In some examples, processing circuitry 250 of server 210 invokes evaluation micro-service 262 in response to a request submitted by the medical device for certain information, which evaluation micro-service 262 may be produce by applying a machine learning model to some combination of the example features described herein. Evaluation micro-service 262 may apply first model 7 to values of configurable settings that are programmed into detection logic of a medical device and based on the application of first model 7, determine whether modified values of the configurable settings, when implemented by the detection logic, would change a determination, by the medical device, regarding whether sensed physiological activity is indicative of cardiac episode for a patient. In response to a determination that the modified settings would change the determination regarding whether the sensed physiological activity is indicative of the cardiac episode for the patient, evaluation micro-service 262 may generate output data indicative of the modified values for the configurable settings for the medical device. To the clinician and/or the patient receiving the modified values, the output data may convey a post-processing (yet online) rejection of the medical device's determination regarding whether sensed physiological activity is indicative of a cardiac episode for the patient; whereas, in other examples, evaluation micro-service 262 further generates output data to express the online rejection, possibly in a textual representation.

In response to a determination that the modified settings would result in the same or similar determination, evaluation micro-service 262 generates output data indicative of a confirmation of the cardiac episode. Because the patient's medical device may be programmed with current or default (i.e., uncalibrated) values of the configurable settings, the output data indicative of the confirmation may further indicate a completed evaluation of the current or default settings values and, in some examples, an approval of those current or default settings for being calibrated to one or more input features.

Monitoring service 6, by way of above evaluation micro-service 262, provides a number of beneficial advantages to medical devices in a medical system or any medical device capable of submitting a service request. To highlight at least one benefit, the patient and clinician, without having to invoke an offline process for the post-processing rejection or confirmation, may utilize (e.g., request) monitoring service 6 to determine whether a possible ailment (e.g., a suspected cardiac episode) is a false positive, false negative, true negative, or a true positive. The patient's medical device may remain online actively recording patient activity (e.g., recording patient cardiac activity) while, in real time, evaluation micro-service 262 determines whether to confirm or reject the possible ailment (e.g., the suspected cardiac episode). Hence, the present disclosure enables real-time/near real time evaluation of patient activity data provided by the patient's medical device.

Monitoring service 6 may operate the example computing service into responding to compatible service requests by returning, to each requesting medical device/external device, a service response delineating the above modified values as well as indicating a rejection/confirmation message. In other examples, monitoring service 6 may operate in an automated manner, for instance, by proactively ensuring medical devices are given an appropriate configuration (e.g., of settings).

Monitoring service 6 may operate the example computing service to perform other functionality. In some examples, processing circuitry 250 of server 210 may (e.g., automatically) configure one or more medical devices with calibrated device settings based on second model 9. Second model 9 may be configured to determine one or more modified values of one or more configurable settings of each medical device where the one or more modified values calibrate the device's configurable settings to a particular output class of (similar) medical devices, the device's type, and/or the patient/patient group. Hence, in some examples, second model 9 may calibrate the medical device to its patient by determining personalized values of the device's configurable settings. In general, second model 9 may include a machine learning resource that patients, via monitoring service 6, may employ to improve their devices, for instance with enhanced accuracy in its detection logic and better overall performance in monitoring and detecting patient maladies.

In other examples, processing circuitry 250 of server 210 invokes evaluation micro-service 262 in response to another request submitted by the medical device for certain information, which evaluation micro-service 262 may be produce by applying another machine learning model, second model 9, to some combination of the example features described herein. Monitoring service 6 may access (e.g., receive or retrieve) current or default values of the medical device's configurable settings and determine, based on the application of second model 9, whether to modify the default or current values of the configurable settings. Evaluation micro-service 262 may generate output data indicative of modified values for the configurable settings for the medical device in response to a determination to modify the default or current values of the configurable settings. If such a modification results in increased or maximized accuracy level and/or other performance metric, monitoring service 6, via server 210, communicates the modified values to the medical device where these values are programmed into the device's logic.

Monitoring service 6 may run rests on models (e.g., machine learning models and mathematical models) to observe model performance in terms or one or more metrics. To run a particular test, processing circuitry 250 of server 210 operates test micro-service 266, which may perform operations on various model data associated with first model 7, second model 9, third model 76, and/or another model or models employed under monitoring service 6. As described herein, third model 76 includes model components/sub-components (e.g., network layers) that are defined by of the medical device's configurable settings; the medical device's detection logic may include an implementation (e.g., in software code) of such model components/sub-components.

One example test may be configured to determine a metric value (e.g., by measuring an accuracy (e.g., level) or computing a performance score) for third model 76 and then, compare that metric value to one or more stored metric values such that comparison results typically distinguish a best performing version of third model 76 from one or more other versions (e.g., default or current versions). It may be assumed that the best performing version of third model 76 includes appropriate values for the device's configurable settings; hence, programming, into the device's detection logic, the appropriate values most likely is to result in maximized monitoring/detection performance scores. As such, the device's configurable settings may be calibrated for a same or similar class of medical devices and the device itself is calibrated for operation on the patient or a similar patient.

Evaluation micro-service 262 may apply second model 9 to feature data of the medical device where the feature data corresponds to a manufacturer of the medical device, a developer of the detection logic, or the patient. Training micro-service 264 may use a variety of features to train second model 9, ultimately, for use in calibrating the medical device's functionality for maximum performance. In this manner, evaluation micro-service 262 may determine appropriate values for the medical device's settings where one or more values are tailored to features of the medical device, the patient, or both. Given a number of different device types, evaluation micro-service 262 may invoke second model 9 to determine a set of values for the configurable settings in each such device. In some examples, monitoring service 6 configures IMD 16 of FIG. 1 with calibrated settings for characteristics (e.g., features) of patient 14 such that IMD 16 is operative to accurate determine whether patient 14's cardiac activity is indicative of a cardiac episode; in such a case, IMD 16 may not be applicable to other patients, especially those unlike patient 14 with respect to personal cardiac activity.

Training micro-service 264 may build fully trained versions of first model 7 and/or second model 9 to include a number of output classes where each class corresponds to devices of a same single type and based on those devices' shared characteristics (e.g., features), training micro-service 264 may train second model 9 to determine a set of values for the configurable settings for each output class. In a supervised learning example, output data (e.g., predictions) of first model 7 may be used to train second model 7. First model 7 may be applied to input features corresponding a suspected cardiac episode and default/current values of an example device's configurable settings, which influence the device's accuracy in episode detection. First model 7 may generate a confirmation of the suspected episode or, as a rejection, modified values for the medical device's configurable settings. The medical device (e.g., IMD 16 or external device 24 on behalf of IMD 16) may provide follow-up data if, for instance, the suspected cardiac episode was rejection but actually occurred.

One example of first model 7 generates output data indicative of modified values for device's configurable settings where implementing these values would cause the medical device to change the determination regarding the suspected cardiac episode in the patient's cardiac activity. The device's determination may be changed to a determination that the patient's cardiac activity is not an episode (e.g., a non-episode) or is a different type of cardiac episode. The changed determination may be more accurate than the device's earlier determination. In other examples, first model 7 generates output data indicative of modified values for the medical device's configurable settings where implementing these values would cause the medical device to change the determination regarding the suspected cardiac episode to a more accurate determination. Again, the updated medical device may change to non-episode or a different type of episode.

Given the above description of the output data generated from first model 7, training micro-service 264 may use that data to train second model 9. A trained version of second model 9 may be configured to determine calibrated values of the medical device's configurable settings based on at least one of the medical device or the patient. In one example, training micro-service 264 may evaluate the modified values described herein for the output data from first model 7 and proceed to determine that one or more of the modified values of the configurable settings are calibrated to at least one of the medical device or the patient. When determining the calibrated values of the device's settings, training micro-service 264 may leave unchanged the above-mentioned one or more modified values (i.e., immutable).

In an unsupervised learning example, test micro-service 266 computes performance scores to differentiate between different sets of settings values, such as those for different features or feature sets (e.g., different manufacturers, different software versions and/or developers, and/or different patient groups). More importantly, some examples of monitoring service 6 may use test results generated by test micro-service 266 in the further training of first model 7 and/or second model 9. In some examples, after determining, for the above medical device's configurable settings, values (e.g., parameter values) that correspond to a highest computed performance score, test micro-service 266 may generate test results data indicating that these values are calibrated based on feature data corresponds to a manufacturer of the medical device, a developer of the detection logic, or the patient/patient group. Training micro-service 264 may extract observed labels from the tests results data and then, use the observed labels to train first model 7 and/or second model 9. During a training process of second model 9, training micro-service 264 may be operative to adjust model components/sub-components such that, based on the above feature data, adjusted second model 9 generates values that are substantially equal to the above observed labels. It should be noted that the generated values represent the device's prediction/expectation of the device's calibrated settings and therefore, are more likely to result in a more accurate determination (e.g., regarding a cardiac episode).

The medical system of claim 1, wherein to generate the output data, the processing circuitry is configured to at least one of determine that at least one of the modified values of the configurable settings is calibrated to at least one of the medical device or the patient, or train a second model using the output data of the model, wherein the second model is configured to determine calibrated values of the configurable settings based on at least one of the medical device or the patient.

In one example, processing circuitry 250 of server 210 generates the above output data based on determining one or more device-agnostic parameter values or one or more device-specific parameter values that are calibrated for maximal performance by the medical device and/or maximum accuracy for the patient.

In one example, processing circuitry 250 of server 210 is configured to generate and then, communicate the output data to suppress an alert mechanism for an initial detection regarding the cardiac episode, wherein the output data comprises instructions for preventing the alert mechanism from operation.

In some examples, monitoring service 6 may assume that third model 76 is static and unable to be changed within server 210 or servers 211. Instead, third model 76 may be configured to generate output data indicative of some prediction/expectation (e.g., of some quantity or quality) corresponding to a determination regarding patient 14's cardiac health; an external device may properly train third model 76 such that third model 76 remains unchanged when uploaded to monitoring service 6 (e.g., in server 210).

To build first model 7 and/or second model 9, processing circuitry 250 of server 210 invokes training micro-service 264 and performs a training process on various (feature) datasets. Among possible features on which to train first model 266, medical device manufacturer, patient history, software developer, firmware version, and/or the like are some examples. Other possible feature may relate to a specific patient or patient group and the particular cardiac activity of the specific patient or patient group. Some combination of these features are implemented in first model 7 and/or second model 9 such that the resulting version built according to training micro-service 264 may be configured to classify medical devices and/or patients into output classes (e.g., device classes) where each output class corresponds to appropriate settings data for a classification of medical device and/or patient. In general, the medical device implements a number of unique and configurable settings of which some or all have at least some influence on the medical device's functionality. The above model may be codified in its corresponding micro-service as described herein.

To illustrate by way of IMD 16 as an example medical device, monitoring service 6 may provide remote support (as a service) to pacemakers, for example, improving an accuracy of suspected cardiac episodes by updating (when possible) IMD 16's configuration as defined herein with a number of settings. IMD 16 may output a service request directed to monitoring service 6 and receive, from server 210, values for these configurable settings that when programmed into detection logic, are most likely to result in an accurate determination of a cardiac episode based on patient 14's cardiac activity data.

If IMD 16 is being put into service for the first time (i.e., an initial configuration), processing circuitry 250 of server 210 communicates the modified settings to replace the medical device's initial default settings; otherwise, the medical device replaces its current settings with the modified settings. As described herein, processing circuitry 250 of server 210 may invoke various micro-services, such as evaluation micro-service 262, to determine values (e.g., parameter values) for IMD 16's configurable settings. When executed by processing circuitry 250 of server 210, a first model micro-service may control access to first model 266, for example, via an interface through which evaluation micro-service 262 and/or training micro-service 264 submit input features corresponding to IMD 16's service request. In some examples, evaluation micro-service 262 invokes first model micro-service 266 to apply a first model (e.g., first model 128A of FIG. 4) to the above features and then, generate modified settings for the medical device's configuration.

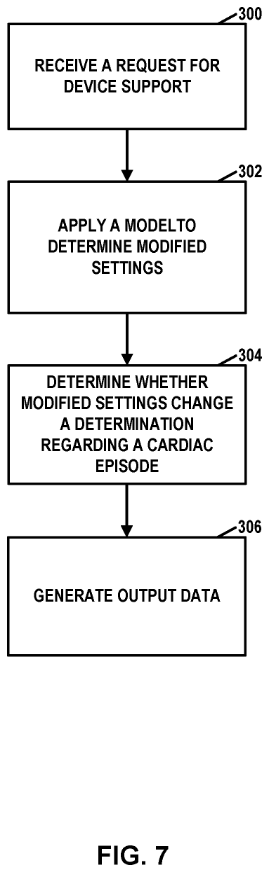

FIG. 7 is a flow diagram illustrating an example operation for remotely monitoring medical devices in an example medical system, such as medical system 10 of FIG. 1, in accordance with one or more examples of the present disclosure.

The example operation illustrated in FIG. 7 may be described within context of FIG. 6, which illustrates server 210 as an example external device such as a server (e.g., server 184 of FIG. 5) in medical system 10 of FIG. 1. A typical medical device of medical system 10 as described herein provides a patient with medical care, for example, by determining whether a cardiac episode occurred in the patient. An example medical device may be implemented with logic (e.g., detection logic) that processes, as input, various datasets corresponding to the patient's physiology/physiological activity and determines whether such patient data indicates a cardiac episode of some type. The patient data may describe sensed physiological activity including patient cardiac activity (e.g., a cardiac EGM). The example medical device may analyze the patient data for cardiac episodes based on various criteria.

For example, as discussed in greater detail with respect to FIGS. 1-2, patient data 72 include cardiac activity data generated by sensing circuitry 82 of IMD 16 and processing circuitry 50 of IMD 16 may monitor patient data 72 for a cardiac episode within the patient cardiac activity, for instance, as depicted in a cardiac EGM. According to the illustrated example of FIG. 6, processing circuitry 250 of server 210 may execute logic configured to host, over a network, an example computing service to assist in (remotely) monitoring IMD 16 or one or more medical devices (e.g., over a network connection) IMD 16 or. The example computing service may be implemented to determine whether a medical device, such as IMD 16 of FIG. 1, is properly configured (e.g., with hardware/software components) for detecting cardiac episodes; if so, the example computing service may perform one or more tasks to ensure that the one or more medical devices can sustain at least an effective level of accuracy.

In the illustrated example of FIG. 7, an example computing service may receive a request for device support from the one or more medical devices (300). In some examples, processing circuitry 250 of server 210 may receive from the one or more medical devices messages requesting the device support in some form and then, run and/or invoke a suitable computing service (e.g., an application and/or data service) for providing that device support.

As directed by that computing service, processing circuitry 250 of server 210 may generate, as a response based on a medical device's request, a communication to include various data including information that, when received, causes a change/update in the requesting medical device's functionality. As described herein, processing circuitry 250 of server 210 may receive a query in the given medical device's request and leverage a resource (e.g., a machine learning resource, such as a machine learning model) to determine appropriate response data for that query.

In some examples, the example computing service may determine whether the one or more medical devices are configured with appropriate settings (e.g., operational settings) for detecting cardiac episodes in general or a specific episode type. An example request (e.g., query) may include various data attributes identifying a requesting device settings and, for example, arranged into patient data that is indicative of a determination (e.g., an initial determination), by the medical device, regarding whether the sensed physiological activity is indicative of a cardiac episode for a patient. In some examples, a given medical device may request operational assistance, for instance, in the form of a post-detection analysis of the patient data that the medical device's request purports to be a suspected cardiac episode by its detection logic. In turn, processing circuitry 250 of server 210 may, on behalf of the example computing service, determine whether the medical device's current settings (or default settings) results in the most likely determination or whether modified settings change the determination into a more/most likely one regarding whether the sensed physiological activity is indicative of the cardiac episode for the patient.

When the above medical device's request is submitted to the example computing service, processing circuitry 250 of server 210 may apply a model to determine modified settings (302) and then, determine whether the modified settings change the initial determination regarding the suspected cardiac episode (304). Depending on a likelihood of the suspected cardiac episode occurring in patient 14, the example computing service may confirm or reject the initial determination; accordingly, processing circuitry 250 of server 210 generates, for communication to the medical device, output data (306) indicating either a confirmation or a rejection of the initial determination regarding the suspected cardiac episode, fulfilling the medical device's request for operational support. When processing circuitry 250 of server 210 communicates the rejection of the initial determination, the medical device may respond by canceling or withholding notifying patient 14. Otherwise, processing circuitry 250 of server 210 communicates the confirmation, and in turn, the medical device may proceed to notify the patient or the patient's clinician by presenting information identifying the suspected cardiac episode.

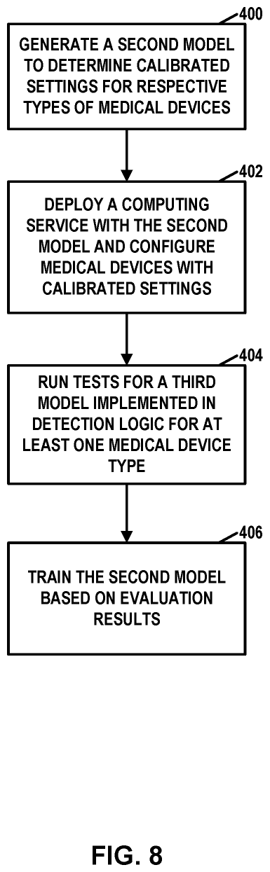

FIG. 8 is a flow diagram illustrating an example operation by a computing service for automatically configuring medical device settings, in accordance with one or more examples of the present disclosure. In some examples, the example operation may be implemented to provide resources in support of medical device functionality and, in some instances, evaluate medical device performance in view of one or more metrics. According to the illustrated example of FIG. 7, processing circuitry 80 of IMD 16 monitors various data (e.g., patient activity data including cardiac activity data) generated by sensing circuitry 82 of IMD 16. For example, as discussed in greater detail with respect to FIGS. 1-2, processing circuitry 50 may monitor patient data 72 for a cardiac episode within the patient activity data, such as a cardiac EGM.

According to the illustrated example of FIG. 6, processing circuitry 250 of server 210 may execute logic configured to host, over a network, an example computing service (e.g., monitoring service 6 of FIGS. 1 and 6) to assist in (remotely) monitoring medical devices, such as IMD 16. The example computing service may utilize training micro-service to first generate a machine learning model to determine settings for respective types of medical devices (400). In some examples, the example computing service may be configured to determine settings that are appropriate for and most likely to detect a cardiac episode for IMD 16 and/or patient 14.

Once generated and initialized, processing circuitry 250 of server 210 may deploy the example computing service to configure similar (e.g., compatible) medical devices to IMD 16 (302). In some examples, the example computing service may perform an automatic configuration to any network-accessible medical device. When IMD 16, for instance, is brought online, processing circuitry 250 of server 210 may recognize IMD 16 and communicate a control directive for configuring IMD 16 with corresponding device settings. In turn, processing circuitry 100 of external device 24 and/or processing circuitry 80 of IMD 16 programs the corresponding device settings into the detection logic (e.g., logic 74) of IMD 16.

In some examples, a medical device may submit a service request to review a determination of a suspected cardiac episode of which the example computing service may determine whether one or more modified settings change the determination of the suspected episode. After handling one or more service requests, processing circuitry 250 of server 210 may train the machine learning model by calibrating IMD 16's configurable settings to features corresponding to IMD 16, patient 14, and/or other factors (304). It is possible for the appropriate settings recommended by the machine learning model to result in a less accurate determination than the initial determination of the suspected cardiac episode; in such instances, the medical device's current/default settings are used to update the machine learning model with one or more learned components (e.g., feature weights). In some examples, processing circuitry 250 of server 210 applies a number of metrics for determining whether the suspected cardiac episode of the initial determination is more or less accurate than the new determination caused by implementing the modified settings recommended by the machine learning model.

In some examples, processing circuitry 250 of server 210 generates a performance score corresponding to the medical device's current or default configurable settings and according to its detection logic configured to generate the determination based on a second machine learning model that corresponds to the current/default settings. The second machine learning model may include various criterion that may be adjusted in accordance with those settings. The performance score is based on one or more metrics. Processing circuitry 250 of server 210 compares the performance score with a second performance score corresponding to the modified settings based on, wherein a modified second machine learning model that corresponds to the modified settings is configured to generate the changed determination. Based on the comparison, processing circuitry 250 of server 210 communicates, to the medical device, the modified settings to update of the detection logic or communicates a confirmation of an initial determination.

In some examples, once trained, the trained machine learning model may be deployed in a second computing service configured to automatically update same or similar medical devices with the calibrated settings (306).

The techniques described in this disclosure may be implemented, at least in part, in hardware, software, firmware, or any combination thereof. For example, various aspects of the techniques may be implemented within one or more microprocessors, DSPs, ASICs, FPGAs, or any other equivalent integrated or discrete logic QRS circuitry, as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, DRAM, SRAM, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

Example 1: A medical system includes processing circuitry configured to: apply a model to values of configurable settings that are programmed into detection logic of a medical device; based on the application of the model, determine whether modified values of the configurable settings, when implemented by the detection logic, would change a determination, by the medical device, regarding whether sensed physiological activity is indicative of cardiac episode for a patient; and in response to a determination that the modified values would change the determination regarding whether the sensed physiological activity is indicative of the cardiac episode for the patient, generate output data indicative of the modified values for the configurable settings for the medical device.

Example 2: The medical system of example 1, wherein the determination regarding whether the sensed physiological activity comprises an initial detection of the cardiac episode based on default values or current values of the configurable settings, wherein to generate the output data, the processing circuitry is configured to: in response to a determination that the modified values would change the initial detection, generate output data indicative of the modified values for the configurable settings for the medical device; and in response to a determination that the modified values would result in the same initial detection, the processing circuitry is further configured to generate output data indicative of a rejection or a confirmation of the initial detection.

Example 3: The medical system of any of examples 1 and 2 further comprising communication circuitry communicatively coupled to the medical device and configured to communicate the modified values of the configurable settings.

Example 4: The medical system of any of examples 1 through 3, wherein to apply the model, the processing circuitry is further configured to apply the model to feature data corresponding to the determination, by the medical device, regarding whether the sensed physiological activity is indicative of cardiac episode for the patient.

Example 5: The medical system of any of examples 1 through 4, wherein to apply the model, the processing circuitry is further configured to apply the model to feature data corresponding to at least one of the medical device or the patient.

Example 6: The medical system of any of examples 1 through 5, wherein to generate the output data, the processing circuitry is configured to at least one of determine that at least one of the modified values of the configurable settings is calibrated to at least one of the medical device or the patient, or train a second model using the output data of the model, wherein the second model is configured to determine calibrated values of the configurable settings based on at least one of the medical device or the patient.

Example 7: The medical system of example 6, wherein to train the second model, the processing circuitry is configured to train the second model using test results for a third model corresponding to the detection logic, wherein a version of the third model is implemented by the detection and is defined by the values of the configurable settings.

Example 8: The medical system of example 7, wherein to train the second model using the test results, the processing circuitry is configured to generate the test results to comprise performance scores corresponding to different versions of the third model.

Example 9: The medical system of any of examples 1 through 8 further includes communication circuitry communicatively coupled to the medical device and configured to receive patient data comprising the physiological activity of the patient sensed by the medical device and indicative of the determination, by the medical device, regarding whether the sensed physiological activity is indicative of the cardiac episode for the patient, wherein the medical device comprises detection logic configured to generate the determination, wherein the values of a plurality of configurable settings are programmed into the detection logic.

Example 10: The medical system of example 9, wherein the communication circuitry is further configured to receive from the medical device a service request for a confirmation or a rejection of the determination.

Example 11: The medical system of any of examples 9 and 10, wherein the processing circuitry, via the communication circuitry, is configured to communicate the modified values of the configurable settings, wherein the medical device is configured to program, into the detection logic, the modified values of the configurable settings.

Example 12: The medical system of any of examples 1 through 11, wherein the medical device comprises at least one of an implantable device, a wearable device, a pacemaker/defibrillator, or a ventricular assist device (VAD) that comprises one or more sensors and sensing circuitry configured to sense the physiological activity.

Example 13: The medical system of any of examples 1 through 12, wherein the processing circuitry is configured to communicate the output data to suppress an alert mechanism for an initial detection regarding the cardiac episode, wherein the output data comprises instructions for preventing the alert mechanism from operation.

Example 14: A method performed by a computing device communicatively coupled to one or more medical devices includes applying, by processing circuitry of the computing device, a model to feature data of the one or more medical devices, wherein the model is configured to calibrate one or more configurable settings of each medical device; by the processing circuitry, determining, based on the application of the model, whether to modify default or current values of the configurable settings; and in response to a determination to modify the default or current values of the configurable settings, generate output data indicative of modified values for the configurable settings for the medical device.

Example 15: The method of example 14, wherein determining further comprises based on the application of the model, determining whether the modified values of the configurable settings, when implemented by the detection logic of the each medical device, would change a determination, by the each medical device, regarding whether sensed physiological activity is indicative of a cardiac episode for a patient.

Example 16: The method of any of examples 14 and 15, wherein generating the output data further comprises determining one or more values for the one or more calibrated settings, wherein the one or more values comprise at least one of one or more device-agnostic parameter values or one or more device-specific parameter values.

Example 17: The method of any of examples 14 through 16, wherein the feature data corresponds to a manufacturer of the medical device, a developer of the detection logic, or the patient.

Example 18: The method of any of examples 14 through 17, wherein generating the output data further comprises communicating the output data to the one or more medical devices, wherein the each medical device is configured to automatically program the modified values into the detection logic in response to the output data.

Example 19: A non-transitory computer-readable storage medium includes apply a model to current or default values of configurable settings that are programmed into detection logic of a medical device, wherein the detection logic is configured to determine whether sensed physiological activity is indicative of cardiac episode for a patient; based on the application of the model, determine whether modified values of the configurable settings, when implemented by the detection logic, would change an initial detection, by the medical device, of the cardiac episode in the sensed physiological activity; and in response to a determination that the modified values would change the initial detection of the cardiac episode for the patient, generate output data indicative of a rejection of the initial detection.

Example 20: The non-transitory computer-readable storage medium of example 19, wherein the instructions that cause the processing circuitry to generate the output data further comprise instructions that cause the processing circuitry to: in response to a determination that the modified values would not change the initial detection of the cardiac episode based on the default or current values of the configurable settings, generate output data indicative of a confirmation of the initial detection.

What is claimed is:

1. A medical system comprising:
processing circuitry configured to:
apply a model to values of configurable settings that are programmed into detection logic of a medical device;
based on the application of the model, determine whether modified values of the configurable settings, when implemented by the detection logic, would change a determination, by the medical device, regarding whether sensed physiological activity is indicative of cardiac episode for a patient; and
in response to a determination that the modified values would change the determination regarding whether the sensed physiological activity is indicative of the cardiac episode for the patient, generate output data indicative of the modified values for the configurable settings for the medical device.

2. The medical system of claim 1,
wherein the determination regarding whether the sensed physiological activity comprises an initial detection of the cardiac episode based on default values or current values of the configurable settings,
wherein to generate the output data, the processing circuitry is configured to:
in response to a determination that the modified values would change the initial detection, generate output data indicative of the modified values for the configurable settings for the medical device; and
in response to a determination that the modified values would result in the same initial detection, the processing circuitry is further configured to generate output data indicative of a rejection or a confirmation of the initial detection.

3. The medical system of claim 1 further comprising communication circuitry communicatively coupled to the medical device and configured to communicate the modified values of the configurable settings.

4. The medical system of claim 1, wherein to apply the model, the processing circuitry is further configured to apply the model to feature data corresponding to the determination, by the medical device, regarding whether the sensed physiological activity is indicative of cardiac episode for the patient.

5. The medical system of claim 1, wherein to apply the model, the processing circuitry is further configured to apply the model to feature data corresponding to at least one of the medical device or the patient.

6. The medical system of claim 1, wherein to generate the output data, the processing circuitry is configured to at least one of determine that at least one of the modified values of the configurable settings is calibrated to at least one of the medical device or the patient, or train a second model using the output data of the model, wherein the second model is configured to determine calibrated values of the configurable settings based on at least one of the medical device or the patient.

7. The medical system of claim 6, wherein to train the second model, the processing circuitry is configured to train the second model using test results for a third model corresponding to the detection logic, wherein a version of the third model is implemented by the detection and is defined by the values of the configurable settings.

8. The medical system of claim 7, wherein to train the second model using the test results, the processing circuitry is configured to generate the test results to comprise performance scores corresponding to different versions of the third model.

9. The medical system of claim 1 further comprising:
communication circuitry communicatively coupled to the medical device and configured to receive patient data comprising the physiological activity of the patient sensed by the medical device and indicative of the determination, by the medical device, regarding whether the sensed physiological activity is indicative of the cardiac episode for the patient, wherein the medical device comprises detection logic configured to generate the determination, wherein the values of a plurality of configurable settings are programmed into the detection logic.

10. The medical system of claim 9, wherein the communication circuitry is further configured to receive from the medical device a service request for a confirmation or a rejection of the determination.

11. The medical system of claim 9, wherein the processing circuitry, via the communication circuitry, is configured to communicate the modified values of the configurable settings, wherein the medical device is configured to program, into the detection logic, the modified values of the configurable settings.

12. The medical system of claim 1, wherein the medical device comprises at least one of an implantable device, a wearable device, a pacemaker/defibrillator, or a ventricular assist device (VAD) that comprises one or more sensors and sensing circuitry configured to sense the physiological activity.

13. The medical system of claim 1, wherein the processing circuitry is configured to communicate the output data to suppress an alert mechanism for an initial detection regarding the cardiac episode, wherein the output data comprises instructions for preventing the alert mechanism from operation.

14. A method performed by a computing device communicatively coupled to one or more medical devices, the method comprising:
applying, by processing circuitry of the computing device, a model to feature data of the one or more medical devices, wherein the model is configured to calibrate one or more configurable settings of each medical device;
by the processing circuitry, determining, based on the application of the model, whether to modify default or current values of the configurable settings; and
in response to a determination to modify the default or current values of the configurable settings, generate output data indicative of modified values for the configurable settings for the medical device.

15. The method of claim 14, wherein determining further comprises based on the application of the model, determining whether the modified values of the configurable settings, when implemented by the detection logic of the each medical device, would change a determination, by the each medical device, regarding whether sensed physiological activity is indicative of a cardiac episode for a patient.

16. The method of claim 14, wherein generating the output data further comprises determining one or more values for the one or more calibrated settings, wherein the one or more values comprise at least one of one or more device-agnostic parameter values or one or more device-specific parameter values.

17. The method of claim 14, wherein the feature data corresponds to a manufacturer of the medical device, a developer of the detection logic, or the patient.

18. The method of claim 14, wherein generating the output data further comprises communicating the output data to the one or more medical devices, wherein the each medical device is configured to automatically program the modified values into the detection logic in response to the output data.

19. A non-transitory computer-readable storage medium comprising program instructions that, when executed by processing circuitry of a medical device, cause the processing circuitry to:
apply a model to current or default values of configurable settings that are programmed into detection logic of a medical device, wherein the detection logic is configured to determine whether sensed physiological activity is indicative of cardiac episode for a patient;
based on the application of the model, determine whether modified values of the configurable settings, when implemented by the detection logic, would change an initial detection, by the medical device, of the cardiac episode in the sensed physiological activity; and
in response to a determination that the modified values would change the initial detection of the cardiac episode for the patient, generate output data indicative of a rejection of the initial detection.

20. The non-transitory computer-readable storage medium of claim 19, wherein the instructions that cause the processing circuitry to generate the output data further comprise instructions that cause the processing circuitry to:
in response to a determination that the modified values would not change the initial detection of the cardiac episode based on the default or current values of the configurable settings, generate output data indicative of a confirmation of the initial detection.
